(19) United States
(12) Patent Application Publication
(10) Pub. No.: US 2008/0057523 A1
(43) Pub. Date: Mar. 6, 2008

(54) Detection of Protein Aggregates by Homologous Elisa

(76) Inventors: Man-Sun Sy, Shaker Heights, OH (US); Tao Pan, Gaithersburg, MD (US); Ruliang Li, Shaker Heights, OH (US)

Correspondence Address: TAROLLI, SUNDHEIM, COVELL & TUMMINO, LLP, 1300 EAST NINTH STREET, SUITE 1700, CLEVELAND, OH, 44114, US

(21) Appl. No.: 11/575,230

(22) PCT Filed: Sep. 16, 2005
(86) PCT No.: PCT/US05/32985

§ 371 Date: Mar. 14, 2007

Related U.S. Application Data
(60) Provisional application No. 60/610,632, filed on Sep. 16, 2004.

(51) Int. Cl. G01N 33/00; G01N 33/53

(52) U.S. Cl. 435/7.92; 435/7.9

(57) ABSTRACT

A method of detecting disease related protein aggregate in a sample comprises using a capture monoclonal antibody and detecting-monoclonal antibody. The capture monoclonal antibody and the detecting monoclonal antibody are identical.

### RELATED APPLICATION

The present application claims priority from U.S. Provisional Application No. 60/610,632 filed Sep. 16, 2004 herein incorporated by reference in its entirety.

### FIELD OF THE INVENTION

The present invention relates to a method of detecting protein aggregates and, more particularly, relates to a capture enzyme-linked immunosorbent assay (ELISA) for detecting protein aggregates.

### BACKGROUND OF THE INVENTION

Assembly or aggregation of conformationally altered proteins is thought to be a major cause of prepathological and pathological conditions including amyloidoses, prion diseases, and other common degenerative diseases. Conformational alterations from α-helical or random coil to β-sheet conformation are believed to be required for the conversion of normally functional proteins into pathogenic states. Examples of proteins capable of conformational changes to form aggregates include: Beta-Amyloid Precursor Protein (APP) and Beta-Amyloid (βA) in amyloid plaques of Alzheimer's Disease (AD), Familial AD (FAD) and cerebral amyloid angiopathy (CAA); α-synuclein deposits in Lewy bodies of Parkinson's disease; Tau in neurofibrillary tangles in frontal temporal dementia and Pick's disease; Superoxide Dismutase in amyotrophic lateral sclerosis; Huntington in Huntington's disease; and Prion Protein (PrP) in Creutzfelds-Jakob disease (CJD) and transmissible spongiform encephalopathy (TSE).

The only diagnostic test for TSE is based on the presence of spongiform histopathology in the brain of affected animals or humans. No reliable in vitro diagnostic test was available until the discovery that TSE or prion disease is caused by the conversion of a normal cellular prion protein, PrP^{C}, into the pathogenic scrapie PrP isoform, PrP^{Sc}. An important effect of the conformational change is that while the entire PrP^{C} is protease-sensitive, the C-terminal domain of PrP^{Sc} becomes relatively protease-resistant. Consequently, the specific detection of protease resistant PrP^{Sc} has provided the basis for the in vitro diagnosis of TSE.

Most often, this specificity has been achieved by the differential proteolysis of PrP^{C} using enzymes, such as proteinase K (PK), prior to the detection of a PK-resistant core of PrP by immunoblotting with an anti-PrP antibody. This procedure can currently detect between 10 and 100 pg (10⁸-10⁹ molecules) of PrP^{Sc}, which is about the same sensitivity as a bioassay of bovine or human tissue for PrP^{Sc} infectivity in mice. More recently, a conformational specific ELISA has also been developed. This assay is based on the observation that upon denaturation, an epitope that is normally buried in PrP^{Sc} is become exposed, and available for antibody binding.

### SUMMARY OF THE INVENTION

The present invention relates to a method of detecting disease-related, pathogenic protein aggregates in a sample. In the method, a first monoclonal antibody or an epitope-binding fragment thereof that is immunoreactive with the protein aggregate is prepared. The first monoclonal antibody or epitope-binding fragment thereof is brought into contact with the sample. Protein aggregates not bound by the first monoclonal antibody or epitope-binding fragment thereof is removed. A second labeled monoclonal antibody or epitope-binding fragment thereof is then brought into contact with the protein aggregate bound by the first monoclonal antibody or epitope-binding fragment thereof. The second labeled monoclonal antibody or epitope-binding fragment thereof comprises a monoclonal antibody or epitope-binding fragment thereof identical to the first monoclonal antibody or epitope-binding fragment thereof. The amount of second labeled monoclonal antibody or epitope-binding fragment thereof bound to the aggregate protein is then detected.

In accordance with an aspect of the invention the method can be used to detect protein aggregate of at least one of beta-amyloid precursor protein (APP), beta-amyloid (βA), α-synuclein protein, tau protein, superoxide dismutase protein, Huntington protein, and prion protein (PrP).

Another aspect of the invention relates to a method of detecting disease related aggregate of prion protein in a sample. In the method, a first monoclonal antibody or an epitope-binding fragment thereof that is immunoreactive with prion protein is prepared. The first monoclonal antibody or epitope-binding fragment thereof is brought into contact with the sample. The prion protein not bound by the first monoclonal antibody is then removed. A second labeled monoclonal antibody or epitope-binding fragment thereof is brought into contact with the prion protein bound to the first monoclonal antibody or epitope-binding fragment thereof. The second labeled monoclonal antibody or epitope-binding fragment thereof comprises a monoclonal antibody or epitope-binding fragment thereof identical to the first monoclonal antibody or epitope-binding fragment thereof. The amount of second labeled monoclonal antibody or epitope-binding fragment thereof bound to the prion protein is then detected.

In an aspect of the invention, the second monoclonal antibody or epitope-binding fragment thereof can be coupled to a T7 polymerase RNA promoter-driven cDNA sequence. The RNA promoter-driven cDNA that can be amplified to detect the prion protein. The amount of second monoclonal antibody or epitope-binding fragment thereof bound to the prion protein can be proportional to the amount of protein aggregate present in the biological sample.

### BRIEF DESCRIPTION OF THE DRAWINGS

FIG. 1 illustrates a schematic flow diagram of an aggregation-specific ELISA in accordance with an aspect of the invention.

FIG. 2 illustrates development of an aggregation-specific ELISA. (A) Presence of PrP dimers in recombinant murine, ovine, bovine, and human PrP. Equal amounts of rMo-PrP, rOv-PrP, rBo-PrP, and rHu-PrP were separated by SDS-PAGE under nonreducing conditions and then immunoblotted with MAb 7A12 (left panel). Dimeric PrP was present in all four preparations of rPrP proteins. Proteins were separated by SDS-PAGE under reducing conditions in the presence of 2-mercaptoethanol (2ME) (right panel). All four rPrP proteins migrated as monomers. Therefore, all four PrP dimers were formed by disulfide bonding. (B). Locales of anti-PrP MAb epitopes. The locales of the anti-PrP MAb epitopes were determined either by using synthetic peptides corresponding to different regions of PrP or by using recombinant PrP fragments. MAb 5C3 reacts with rHu-PrP₂₃₋₁₄₅ as well as rHu-PrP₉₀₋₂₃₀. MAbs 6H3, 7C11, 12H7, and 8C6 react with rHu-PrP₉₀₋₂₃₀ but do not react with rHu-PrP₂₃₋₁₄₅. These MAbs do not react with any synthetic peptides. (C). Identification of MAbs that react preferentially with rMo-PrP, rOv-PrP, rBo-PrP, and rHu-PrP. Conventional ELISA plates were precoated with 0.5 μg/well of affinity-purified anti-PrP MAbs (the MAbs were numbered from 1 to 30). Different rPrP proteins (2.5 ng/well) were then added into each well. After three washes, a biotinylated MAb that is identical to the precoated, capture MAb was added to detect dimeric PrP as described in the text. Only a minority of the tested MAbs could detect rPrP dimers. Some MAbs, such as 7A12 and 11G5, that react with rMo-PrP also react with rOv-PrP and rBo-PrP as well as rHu-PrP dimers. Nevertheless, there are MAbs that preferentially react with rPrP dimer in a species-specific manner. Hence, the four mammalian rPrP dimers share common features, but each also has unique features.

FIG. 3 illustrates an aggregation-specific ELISA is dimer specific (A and B). Different concentrations of rMo-PrP were separated by SDS-PAGE and then immunoblotted with MAb 7A12 as described in the text. Only preparation A contains dimeric PrP. (C and D) The amount of rPrP in each preparation was quantified using a conventional ELISA, in which MAb 8B4 was used as the capture MAb and biotinylated MAb 7A12 was used as the detecting MAb. It is clear that the two preparations of rMo-PrP contained similar amounts of total rPrP protein. The amount of dimeric PrP in each preparation was also quantified using MAb 8B4/8B4 and MAb 8H4/8H4 (as negative controls) and MAb 11G5/11G5 and MAb 7A12/7A12 to detect dimers. Only preparation A, which has dimeric PrP, has MAb 11G5/11G5 and MAb 7A12/7A12 immunoreactivity.

FIG. 4 identifies MAbs that react preferentially with PrP aggregates in ME7-infected brains. (A). Comparison between recombinant mouse PrP dimers and PrP^{Sc} aggregate in ME7-infected brains. Conventional ELISA plates were precoated with 0.5 μg/well of affinity-purified anti-PrP MAbs (numbers 1 to 30). Three microliters of a 20% brain homogenate (containing 60 μg of total brain proteins, without proteinase K treatment) from ME7-infected mice at terminal stages of disease was then added into each well. After three washes, a biotinylated MAb that is identical to the precoated, capture MAb was added to detect PrP aggregates. Only one of the five MAbs, 11G5, which reacts with rMo-PrP dimer, also reacted with PrP aggregates present in PrP^{Sc}-infected mouse brains. Three MAbs, 6H3, 8F9, and 7H6, that reacted with infected brain homogenates did not react with rMo-PrP dimer. Hence, the PrP^{Sc} aggregates formed in vivo and the recombinant PrP dimer generated in vitro have different conformations or epitope accessibilities. (B). Sensitivity of the aggregation-specific ELISA and the specificity of the detecting MAb. Left panel: different concentrations of total brain proteins were added onto the ELISA plates that had been precoated with MAb 11G5. A biotinylated 11G5 was then used to detect bound PrP. The assay detects signal in sample that contains approximately 6 μg of total brain proteins. Right panel: 60 micrograms of total brain proteins (without proteinase K treatment) was added onto the ELISA plates that had been precoated with MAb 11G5. Prior to adding biotinylated 1G5, different amounts of either unconjugated MAb 8H4 or unconjugated MAb 11G5 were added onto the well. It is clear that only unconjugated MAb 11G5 can block the binding of biotinylated 11G5 in an antibody concentration-dependent manner. Therefore, the detecting MAb is epitope specific.

FIG. 5 illustrates that most of the PrP aggregates detected by the aggregation-specific ELISA are present in fractions 3, 4, and 5. One sham-infected control and one ME7-infected brain homogenate were fractionated in a 10-to-60% sucrose gradient as described in the text. Each fraction was immunoblotted with MAb 8H4. (A). All the PrP^{C} in the sham-infected control is in the top fractions, 1 and 2. (B). In infected brain, immunoreactivity is present in all fractions; however, fractions 10 and 11 have the highest levels of immunoreactivity. (C). The presence of PrP aggregates in each fraction was also quantified by the aggregation-specific ELISA using MAb 11G5/11G5. This antibody pair did not detect any immunoreactivity in any fraction from sham-infected control brain homogenate. In contrast, significant immunoreactivity was detected in fractions 3, 4, and 5 from ME7-infected brain homogenates. *, significant difference between identical fractions from infected brains and control brains (P 0.005).

FIG. 6 illustrates a schematic view of an aggregate-specific ELISA. In PrP aggregates, a defined epitope may have multiple presences (right), while in the PrP monomer it is only presented once (left). Therefore, by using one single MAb as both capture and detecting antibody, PrP aggregates can be distinguished from monomers.

FIG. 7 illustrates AS-FACT is more sensitive than AS-ELISA in detecting rPrP dimers. (A). AS-ELISA: ELISA plates were coated with MAb 11G5. Various concentrations of rMo-PrP, ranging from 1 ng/ml to 2 00 ng/ml (0.1 ml/well) were added to the plates in duplicates. A biotinylated MAb-11G5 was then added to react with the bound rPrP. AS-ELISA can detect rPrP at 20 ng/ml, which corresponds to 2 ng of rPrP. The results presented were the average of the duplicates +1-S.E. (B). AS-FACT: 368 well plates were coated with MAb 11G5 (0.5 μg/ml, 20 μl/well in 0.05M carbonate-bicarbonate buffer, pH 9.6. Different concentrations of rMo-PrP, ranging from 0.01 ng/ml to 1000 ng/ml (0.02 ml/well) were added to the plates in duplication. A biotinylated MAb-11G5 was then added to react with the bound rPrP. T7 amplification was carried out as described in Example 2. AS-FACT could detect rPrP at 0.01 ng/ml, which corresponds to 2 pg of rPrP. The results presented were the average of the duplicates +/− S.E.

FIG. 8 illustrates AS-FACT is also more sensitive than AS-ELISA in detecting PrP^{Sc} aggregates in infected brains (A). AS-ELISA: Individual brain homogenate (20%, W/V) from either non-infected control mice (n=2) or from ME7-infected, terminally ill mice (n=2) was serially diluted in PBS. 0.1 ml/well of the each dilution was added to ELISA plates pre-coated with MAb 11G5 in duplicates. A biotinylated MAb-11G5 was then added to react with the bound PrP. Both infected brains have much higher immunoreactivity at undiluted and samples diluted after 1:10 dilution, the differences between infected and non-infected homogenates were statistically insignificant. The results presented were the average of the duplicates +/− S.E. from individual mouse. (B). AS-FACT: 384 well plates were coated with MAb 11G5. Serially diluted brain homogenates (from a 20% W/V) were added to the plates in duplicates. A biotinylated MAb-11G5 was then added to react with the bound rPrP. T7 amplification was carried out as described in Example 2. AS-FACT could detect rPrP at 0.01 ng/ml, which corresponds to 2 pg of rPrP. The results presented were the average of the duplicates +/− S.E.

FIG. 9 illustrates the temporal appearance of PrP in brain of mice infected intraperitoneally with ME7 PrP^{Sc} (A). PrP^{Sc} aggregates could be detected as early as 7 days after an intraperitoneally inoculation: individual brain homogenates (containing approximately 4 μg/ml of total brain proteins) from normal non-infected mice, mice injected intraperitoneally with normal brain homogenates 24 hrs earlier, PrP^{C-/-} (“knock-out”) mice or mice infected intraperitoneally with ME7 PrP^{Sc} at various time points was assay for PrP^{Sc} by AS-FACT. There is a significant difference in immunoreactivity between normal and PrP^{C-/-} brain homogenates, indicating a small amount of PrP aggregates is present in normal brain. There is not a difference among non-infected control mice, mice injected with normal brain homogenates 24 hrs earlier, or mice injected 24 hrs earlier with infected brain homogenates. There are significant differences between normal controls and mice injected with PrP^{Sc} 7 days or 21 days earlier.

Compared are normal to PrP^{C-/-}, P=0.039; normal to mice injected with normal brain homogenates 24 hrs earlier, P=0.298; normal to mice injected with PrP^{Sc} brain homogenates 24 hrs earlier, P=0.08; normal to mice injected with PrP^{Sc} one week earlier, P<0.0001, three weeks earlier, P<0.0001, five weeks earlier P<0.0001; mice injected one week earlier and mice injected three weeks earlier, P=0.335; mice injected three weeks earlier and mice injected five weeks earlier, P<0.0001. (B). Temporal appearance of PrP^{Sc} during the disease progression: mice were inoculated with ME7 PrP^{Sc} intraperitoneally. At different days after inoculation, individual brain homogenate was prepared and assayed for PrP^{Sc} aggregates by AS-FACT. Fluorescent readings from a group of five normal mice were averaged and used as background. Results presented were the average +/− S.E. of at least 5 mice for each time point with the background subtracted.

FIG. 10 illustrates the detection of PrP aggregates in CWD brains (A). AS-ELISA: Individual brain homogenates (containing 600 μg/ml of total brain proteins) from either non-CWD animals (n=6) or CWD animals (n=3) were assayed in AS-ELISA using MAb 11G5. All three CWD animals have slightly but significant higher immunoreactivity. (B). AS-FACT: Individual brain homogenates (containing 4 μg/ml of total brain proteins) from either non-CWD animals (n=6) or CWD animals (n=3) were assayed in AS-FACT using MAb 11G5. All three CWD animals have much higher immunoreactivity.

FIG. 11 illustrates the detection of PrP^{Sc} aggregates in vCJD by AS-ELISA and AS-FACT (A). AS-ELISA with MAb 11G5: Individual brain homogenates (containing 600.μg/ml of total brain proteins) from non-CJD controls (n=8), vCJD (n=8) or sCJD (n=8) were assayed in AS-ELISA using MAb 11G5. All samples from vCJD reacted stronger than samples from non-CJD controls (P=0.002) or from sCJD. (B). AS-ELISA with MAb 6H3: Identical to (A), except MAb 6H3 was used. Again, all vCJID samples have much stronger immunoreactivity than controls (P=0.0001). (C). AS-FACT with either MAb 11G5 or 6H3: Individual brain homogenates (containing 4 μg/ml of total brain proteins) from non-CJD controls (n=8), vCJD (n=8) or sCJD (n=8) were assayed in AS-FACT using either MAb 11G5 or 6H3. The immunoreactivities in vCJD were significantly higher than controls (11G5, P=0.0001; 6H3, P=0.0001) and sCJD. (D). AS-FACT with MAb 6H3: Two vCJD samples were serially diluted and assayed for PrP^{Sc} aggregates in AS-FACT. The assay detects signals even at 0.08 μg/ml of total brain proteins in both samples.

### DETAILED DESCRIPTION

The present invention relates to a novel aggregation specific (AS)-ELISA (AS-ELISA) that can be used to detect aggregates of conformationally-altered proteins. (i.e., abnormal protein aggregation) in a biological sample. Proteins, such as beta-amyloid precursor protein (APP), beta-amyloid (βA), α-synuclein protein, tau protein, superoxide dismutase protein, Huntington protein, and prion protein (PrP), can be capable of changing their conformation to form disease-related, pathogenic aggregates. These aggregates can be a major cause of prepathological and pathological conditions including Alzheimer's disease (AD), familial AD (FAD), and cerebral amyloid angiopathy (CAA) (APP and αA aggregates), Parkinson's disease (α-synuclein aggregates), frontal temporal dementia and Tick's disease (tau aggregates), amyotrophic lateral sclerosis (superoxide dismutase aggregates), Huntington's disease (Huntington aggregates), and Creutzfelds-Jakob disease (CJD) and transmissable spongiform encephalopathy (TSE) (prion protein aggregates). The proteins, which form the disease related aggregates, can have monoclonal antibody (MAb) binding epitopes that upon aggregation of the proteins be buried so that none of the binding epitopes are detectable or can be present more than once so that multiple binding epitopes are available for binding.

In an AS-ELISA in accordance with an aspect of the present invention, identical MAbs are used as a capture-MAb as well as a detecting-MAb. The MAb can bind to a single binding epitope that is expressed by a protein of interest. When a biological sample containing normal monomer proteins that express one binding epitope per monomer is brought into contact with immobilized capture MAbs, the capture MAb can bind to the binding epitope of each monomer. When detection of the captured normal monomer protein is made using a detecting-MAb that comprises a MAb identical to the capture-MAb, the detecting-MAb will not bind to the protein because the binding epitope is already occupied. When the conformation of a protein is altered, it may aggregate, in this situation, the same binding epitope can be expressed more than once. Multiple detecting-MAb can bind to each aggregate to detect the abnormal protein aggregate and the presence of the disease associated with the abnormal protein aggregation. For example, the AS-ELISA can be used to detect disease-related aggregates of PrP^{Sc} in the brain of mice at about 70 days post-intracerebral inoculation, at a time when no protease resistant PrP^{Sc} is detectable.

The term “detecting” in accordance with the present invention is used in the broadest sense to include both qualitative and quantitative measurements of abnormal protein aggregation. In one aspect, the detecting method as described herein is used to identify the mere presence of disease-related protein aggregate in a biological sample. In another aspect, the method is used to test whether disease related protein aggregate in a sample is at a detectable level. In yet another aspect, the method can be used to quantify the amount of disease related protein aggregates in a sample and further to compare the amount of aggregates in different samples The term “biological sample” refers to a body sample from any animal, but preferably is from a mammal, more preferably from a human. Such samples include biological fluids, such as serum, plasma, vitreous fluid, lymph fluid, synovial fluid, follicular fluid, seminal fluid, amniotic fluid, milk, whole blood, urine, cerebro-spinal fluid, saliva, sputum, tears, perspiration, mucus, and tissue culture medium, as well as tissue extracts, such as homogenized tissue, and cellular extracts.

The term “capture monoclonal antibody” refers to an antibody that is capable of binding and capturing an protein aggregate in a sample such that under suitable condition, the capture antibody-protein aggregate complex can be separated from the rest of the sample. Typically, the capture monoclonal antibody is immobilized or immobilizable.

The term “detecting-monoclonal antibody” or “detectable monoclonal antibody” refers to a monoclonal antibody that is capable of being detected either directly through a label amplified by a detection means, or indirectly through, e.g., another antibody that is labeled. For direct labeling, the antibody is typically conjugated to a moiety that is detectable by some means. The preferred detectable monoclonal antibody is biotinylated monoclonal antibody.

The term “detection means” refers to a moiety or technique used to detect the presence of the detectable monoclonal antibody in the ELISA herein and includes detection agents that amplify the immobilized label such as label captured onto a microtiter plate.

The term “antibody” is used in the broadest sense and includes monoclonal antibodies (including agonist, antagonist, and neutralizing antibodies) and epitope-binding antibody fragments thereof so long as they exhibit the desired binding specificity.

The term “monoclonal antibody” as used herein refers to an antibody obtained from a population of homogeneous antibodies, i.e., the individual antibodies comprising the population are identical except for possible naturally-occurring mutations that may be present in minor amounts. Monoclonal antibodies are highly specific, being directed against a single epitope binding site. Furthermore, in contrast to conventional (polyclonal) antibody preparations that typically include different antibodies directed against different determinants (epitopes), each monoclonal antibody is directed against a single determinant on the protein. The modifier “monoclonal” indicates the character of the antibody as being obtained from a homogeneous population of antibodies, and is not to be construed as requiring production of the antibody by any particular method. For example, the monoclonal antibodies to be used in accordance with the present invention may be made by the hybridoma method first described by Kohler et al. Nature 256:495 (1975), or may be made by recombinant DNA methods (see, e.g., U.S. Pat. No. 4,816,567, herein incorporated by reference in its entirety). The “monoclonal antibodies” may also be isolated from phage antibody libraries using the techniques described in Clackson et al Nature 352:624-628 (1991) and Marks et al. J. Mol. Biol. 222:581-597 (1991), for example.

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al. Proc. Natl. Acad. Sci. USA 81:6851-6855 (1984)).

“Humanized” forms of non-human (e.g., murine) antibodies are chimeric antibodies that contain minimal sequence derived from non-human immunoglobulin. For the most part, humanized antibodies are human immunoglobulins (recipient antibody) in which hypervariable region residues of the recipient are replaced by hypervariable region residues from a non-human species (donor antibody), such as mouse, rat, rabbit or nonhuman primate having the desired specificity, affinity, and capacity. “Mammal” for purposes of treatment refers to any animal classified as a mammal, including humans, domestic, and farm animals, and zoo, sports, or pet animals, such as dogs, horses, cats, sheep, pigs, cows, etc. Preferably, the mammal is human.

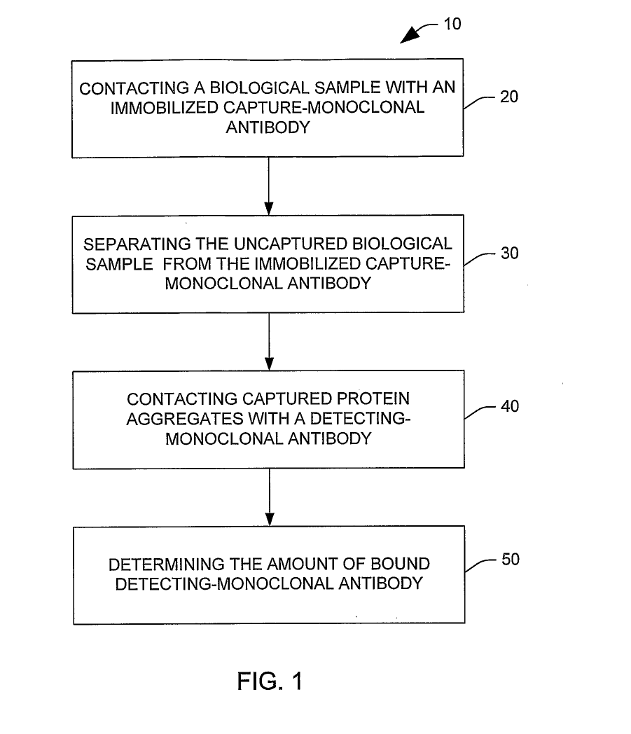

FIG. 1 is schematic flow diagram illustrating a method 10 of using the AS-ELISA in accordance with an aspect of the invention. In the method 10, at 20, a biological sample is contacted with an immobilized capture-monoclonal antibody (or epitope-binding fragment thereof). Monoclonal antibodies of the present invention can be selected that are immunoreactive with or capable of binding to a binding epitope that is expressed once on a normal protein monomer and can be potentially expressed multiple times in a conformationally altered disease related aggregates of the protein monomer. Optionally, monoclonal antibodies of the present invention can be selected that are capable of binding to binding epitopes that are expressed more than once on a normal protein monomer and that can be suppressed (e.g., blocked or buried) in a conformational altered disease related aggregate of the protein monomer.

The monoclonal antibodies that are capable of binding to epitopes of the proteins can be from any species, such as murine. The monoclonal antibodies can be produced by known monoclonal antibody production techniques. Typically, monoclonal antibodies are prepared by recovering spleen cells from immunized animals with the protein of interest and immortalizing the cells in conventional fashion, for example, by fusion with myeloma cells or by Epstein-Barr virus transformation, and screening for clones expressing the desired antibody. See, for example, Kohler and Milstein Eur. J. Immunol. 6:511 (1976). Monoclonal antibodies, or the epitope-binding region of a monoclonal antibody, may alternatively be produced by recombinant methods.

By way of example, where the protein of interest is a prion protein that is capable of changing it's conformation to form PrP^{Sc} aggregates, the monoclonal antibody can be a murine monoclonal antibody that is generated by immunizing “knock out” mice with recombinant normal mouse cellular protein (PrP^{c}). Spleen cells (antibody producing lymphocytes of limited life span) from the immunized mice can then be fused with non-producing myeloma cells (tumor lymphocytes that are “immortal”) to create hybridomas. The hybridomas can then be screened for the production of antibody specific to prion and the ability to multiply indefinitely in tissue culture. These hybridons can then be propagated to provide a permanent and stable source for the specific monoclonal antibodies. Particular monoclonal antibodies produced by this method are disclosed in U.S. Pat. No. 6,528,269, which is herein incorporated by reference in its entirety.

Monoclonal antibodies disclosed in U.S. Pat. No. 6,528,269, such as 2F8, 5B2, 6H3, 8C6, 8H4 and 9H7 produced by cell lines PrP2F8, PrP5B2, PrP6H3, PrP8C6, PrP8H4 and PrP9H7, can recognize not only human prion protein, but they can be cross-reacted with prion proteins from mouse, cow, sheep and other species. These antibodies are believed to be the first panel of monoclonal antibodies that are capable of reacting with human, mouse, sheep and cow prion proteins.

The capture-monoclonal antibody (e.g., 6H3) can be immobilized on a solid phase by insolubilizing the capture-monoclonal antibody before the assay procedure, as by adsorption to a water-insoluble matrix or surface (U.S. Pat. No. 3,720,760, herein incorporated by reference in its entirety) or non-covalent or covalent coupling, for example, using glutaraldehyde or carbodiimide cross-linking, with or without prior activation of the support with, e.g., nitric acid and a reducing agent as described in U.S. Pat. No. 3,645,852 or in Rotmans et al., J. Immunol. Methods 57:87-98 (1983)), or afterward, such as by immunoprecipitation.

The solid phase used for immobilization may be any inert support or carrier that is essentially water insoluble and useful in immunometric assays, including supports in the form of, for example, surfaces, particles, porous matrices, etc. Examples of commonly used supports include small sheets, Sephadex, polyvinyl chloride, plastic beads, and assay plates or test tubes manufactured from polyethylene, polypropylene, polystyrene, and the like including 96-well microtiter plates and 384-well microtiter well pates, as well as particulate materials, such as filter paper, agarose, cross-linked dextran, and other polysaccharides. Alternatively, reactive water-insoluble matrices, such as cyanogen bromide-activated carbohydrates and the reactive substrates described in U.S. Pat. Nos. 3,969,287; 3,691,016; 4,195,128; 4,247,642; 4,229,537; and 4,330,440 are suitably employed for capture-monoclonal antibody immobilization. In one example, the immobilized capture-monoclonal antibodies are coated on a microtiter plate, and in particular the preferred solid phase used is a multi-well microtiter plate that can be used to analyze several samples at one time. For example, the multi-well microtiter plate can be a microtest 96-well ELISA plate, such as that sold by Nune Maxisorb or Immulon.

The solid phase is coated with the capture-monoclonal antibody (e.g., 6H3), which may be linked by a non-covalent or covalent interaction or physical linkage as desired. Techniques for attachment include those described in U.S. Pat. No. 4,376,110 and the references cited therein. If covalent binding is used, the plate or other solid phase can be incubated with a cross-linking agent together with the capture reagent under conditions well known in the art.

Commonly used cross-linking agents for attaching the capture-monoclonal antibody to the solid phase substrate include, e.g., 1,1-bis(diazoacetyl)-2-phenylethane, glutaraldehyde, N-hydroxysuccinimide esters, esters with 4-azidosalicylic acid, homobifunctional imidoesters, including disuccinimidyl esters such as 3,3′-dithiobis(succinimidylpropionate), and bifunctional maleimides such as bis-N-maleimido-1,8-octane. Derivatizing agents, such as methyl-3-[(p-azidophenyl)dithio]propioimidate yield photoactivatable intermediates capable of forming cross-links in the presence of light.

If micro-titer well plates (e.g., 96-well plates or 384-well plates) are utilized, they can be coated with the affinity purified capture monoclonal antibodies (typically diluted in a buffer) at, for example, room temperature and for about 2 to about 3 hours. The plates may be stacked and coated long in advance of the assay itself, and then the assay can be carried out simultaneously on several samples in a manual, semi-automatic, or automatic fashion, such as by using robotics.

The coated plates are then typically treated with a blocking agent that binds non-specifically to and saturates the binding sites to prevent unwanted binding of the free ligand to the excess sites on the wells of the plate. Examples of appropriate blocking agents for this purpose include, e.g., gelatin, bovine serum albumin, egg albumin, casein, and non-fat milk.

After coating and blocking, a biological sample comprising the protein to be analyzed is added to the immobilized phase. The biological sample can be appropriately diluted with, for example, a lysis buffer (e.g., phosphate-buffered saline (PBS) with 1% Nonidet P-40, 0.5% sodium deoxycholate, 5 mM EDTA, and pH 8.0).

For sufficient sensitivity, the amount of biological sample added to the immobilized capture monoclonal antibody can be such that the immobilized capture monoclonal antibodies are in molar excess of the maximum molar concentration of the conformational altered protein anticipated in the biological sample after appropriate dilution of the sample. This anticipated level depends mainly on any known correlation between the concentration levels of the protein in the particular biological sample being analyzed with the clinical condition of the patient.

The conditions for incubation of the biological sample and immobilized monoclonal antibody are selected to maximize sensitivity of the assay and to minimize dissociation. Preferably, the incubation is accomplished at fairly constant temperatures, ranging from about 0° C. to about 40° C., such as room temperature (e.g., about 25° C.). The time for incubation depends primarily on the temperature, being generally no greater than about 10 hours to avoid an insensitive assay. For example, the incubation time can be from about 0.5 to 3 hours, and particularly about 1.5 to about 3 hours at room temperature to maximize binding to the capture monoclonal-antibodies.

Following contact of the biological sample the immobilized capture-monoclonal antibody (e.g., 6H3), at 20, the biological sample is separated (preferably by washing) from the immobilized capture-monoclonal antibodies to remove uncaptured proteins. The solution used for washing is generally a buffer (“washing buffer”) with a pH determined using the considerations and buffers typically used for the incubation step. The washing may be done, for example, three or more times. The temperature of washing is generally from refrigerator to moderate temperatures, with a constant temperature maintained during the assay period, typically from about 0 to about 40° C. Optionally, a cross-linking agent or other suitable agent may be added at this stage to allow the now-bound protein to be covalently attached to the capture monoclonal antibodies if there is any concern that the captured proteins may dissociate to some extent in the subsequent steps.

Following separation of the uncaptured biological sample, at 30, the immobilized capture-monoclonal antibodies (e.g., 6H3) and captured protein aggregates are contacted with detecting-monoclonal antibodies (or epitope-binding fragments thereof), at a temperature, for example, of about 20° C. to about 40° C. The detecting-monoclonal antibody comprises a monoclonal antibody that is identical to the capture monoclonal antibody. By identical it is meant that the detecting-monoclonal antibody is obtained from a population of homogeneous monoclonal antibodies, i.e., the individual antibodies comprising the population are identical except for possible naturally-occurring mutations that may be present in minor amounts. A molar excess of the detecting-monoclonal antibody with respect to the maximum concentration of free binding epitopes expected is added to the plate after it is washed.

The detecting-monoclonal antibody can be labeled with any detectable functionality that does not interfere with the binding of the detecting-monoclonal antibody to free binding epitopes on the bound proteins. Examples of suitable labels are those numerous labels known for use in immunoassays, including moieties that may be detected directly, such as fluorochrome, chemiluminescent, and radioactive labels, as well as moieties, such as enzymes, that must be reacted or derivatized to be detected. Examples of such labels include the radioisotopes ³²P, ¹⁴C, ¹²⁵I, ³H, and ¹³¹I, fluorophores, such as rare earth chelates or fluorescein and its derivatives, rhodamine and its derivatives, dansyl, umbelliferone, luceriferases, e.g., firefly luciferase and bacterial luciferase (U.S. Pat. No. 4,737,456), luciferin, 2,3-dihydrophthalazinediones, horseradish peroxidase (HRP), alkaline phosphitase, β-galactosidase, glucoamylase, lysozyme, saccharide oxidases, e.g., glucose oxidase, galactose oxidase, and glucose-6-phosphate dehydrogenase, heterocyclic oxidases such as uricase and xanthine oxidase, coupled with an enzyme that employs hydrogen peroxide to oxidize a dye precursor such as HPP, lactoperoxidase, or microperoxidase, biotin/avidin, biotin/streptavidin, biotin/Streptavidin-β-galactosidase with MUG, spin labels, bacteriophage labels, stable free radicals, and the like.

Conventional methods are available to bind these labels covalently to proteins or polypeptides. For instance, coupling agents, such as dialdehydes, carbodiimides, dimaleimides, bis-imidates, bis-diazotized benzidine, and the like may be used to tag the antibodies with the above-described fluorescent, chemiluminescent, and enzyme labels, e.g., U.S. Pat. Nos. 3,940,475 (fluorimetry) and 3,645,090 (enzymes); Hunter et al Nature 144:945 (1962); David et al. Biochemistry 13:1014-1021 (1974); Pain et al. J. Immunol. Methods 40:219 230 (1981); and Nygren J. Histochem and Cytochem 30:407-412 (1982).

The conjugation of such label, including the enzymes, to the antibody is a standard manipulative procedure for one of ordinary skill in immunoassay techniques. See, for example, O'Sullivan et al. “Methods for the Preparation of Enzyme-antibody Conjugates for Use in Enzyme Immunoassay,” in Methods in Enzymology, ed. J. J. Langone and H. Van Vunakis, Vol. 73 (Academic Press, New York, N.Y., 1981), pp. 147-166.

Following the addition of detecting-monoclonal antibody, at 40, the amount of bound detecting-monoclonal antibody is determined by removing excess unbound labeled monoclonal antibody by washing and then measuring the amount of the attached label using a detection method appropriate to the label. For example, in the case of enzymes, the amount of color developed and measured can be a direct measurement of the amount of protein aggregates present.

The amount of protein aggregates present can be quantified using known ELISA quantification methods, such as comparing the detected protein aggregates with a standard or by comparing serial diluted samples. Where the monoclonal antibody used with the AS-ELISA is directed to a binding epitope that is expressed only once on a normal monomer, the absence of substantially any detectable signal can be indicative of the absence of substantially any disease related, aggregates of the protein. Conversely, with the same assay, the measurement of a detectable signal can be indicative of the presence of disease related aggregates of the protein, the level of the detected signal being proportional to the amount protein aggregates in the sample. On the other hand, the absence of any detectable signal can be indicative of the disease related aggregates of the protein as well, as binding epitopes may potentially be buried by aggregation of the protein monomers. In this case, the signal detected in the disease related protein aggregate will be lowered than the signal detected in the normal protein monomer.

In accordance with another aspect of the invention, the sensitivity of the AS-ELISA can be substantially increased by employing amplification techniques. The detecting-monoclonal antibody can be coupled to a RNA promoter-driven cDNA sequence, such as disclosed in U.S. Pat. No. 5,922,553, herein incorporated by reference in its entirety. The RNA promoter-driven cDNA sequence, (e.g., T7), coupled to the detecting-monoclonal antibody can be amplified using an RNA polymerase (e.g., T7 RNA polymerase). The amount of amplified product is determined by quantifying levels of the promoter driven cDNA sequence covalently coupled to the bound detecting-monoclonal antibody via the amplified RNA technique. This technique can result in greater sensitivity (e.g., 1,000 times greater sensitivity than AS-ELISA) when used in conjunction with the AS-ELISA of the present invention.

The following examples are intended to illustrate one embodiment now known for practicing the invention, but the invention is not to be considered limited to these examples. All open and patented literature citations herein are expressly incorporated by reference.

### EXAMPLES

### Example 1

### An Aggregation-Specific Enzyme-Linked Immunosorbent Assay: Detection of Conformational Differences between Recombinant PrP Protein Dimers and PrP^{Sc} Aggregates

In this series of examples, we describe the development of a novel enzyme-linked immunosorbent assay (ELISA) that reacts specifically with PrP dimers or PrP aggregates. We used this assay to compare dimeric PrP from four mammalian species, murine, ovine, bovine, and human. Furthermore, we describe the use of this assay to determine whether similar dimeric or distinct PrP aggregates are present in brain homogenates from normal or PrP_{Sc}-infected mice and discuss the nature of these PrP species.

Materials and Methods

Recombinant PrP Proteins

The generation of recombination PrP proteins from different mammalian species has been described in Brown et al., “Functional and structural differences between the prion protein from two alleles prnpa and pmpb of mouse.” Eur. J. Biochem. (2000) 267:2452-2459; and Swietnicki, et al. “pH dependent stability and conformation of the recombinant human prion protein PrP” (90-231). J. Biol. Chem. 272:27517-27520; Yin et al. “On-column purification and refolding of recombinant bovine prion protein: using its octarepeat sequences as a natural affinity tag.” Protein Expr. Purif. (2003) 32:104-109.

Anti-PrP^{C} MAbs

The generation and characterization of anti-PrP^{C} monoclonal antibodies (MAbs) has been described in U.S. Pat. No. 6,528,269 and Sy et al. “Identification of an epitope in the C terminus of normal prion protein whose expression is modulated by binding events in the N-terminus.” J. Mol. Biol. (2000) 301:567-574. All MAbs were affinity purified with protein G chromatography. MAbs were biotinylated using the EZ-Link sulfo-NHS-biotin kit (Pierce Endogen, Rocklford, EL).

Mice

ME7, 139A, or 22L mouse-adapted scrapie strains were propagated by intracerebral injection into 7-week-old CD-1 (Prnp^{a}) mice as described in U.S. Pat. No. 6,528,269. Unless stated, all the animals were sacrificed at the terminal stage of the disease. For ME7 and 139A, this was approximately 170 days postinoculation, and for 22L it was approximately 140 days postinoculation. Sham-infected, age- and sex-matched CD-1 mice were used as controls. All animal experiments were carried out according to institutional regulations and standards.

Preparation of Brain Homogenate.

To prepare 20% (wt/vol) total brain homogenate, individual entire brain samples were homogenized in ice-cold lysis buffer (phosphate-buffered saline [PBS] with 1% Nonidet P-40, 0.5% sodium deoxycholate, 5 mM EDTA; pH 8.0) in the presence of 1 mM phenylmethylsulfonyl fluoride (PMSF; Sigma, Mo.). If the homogenate was to be treated with proteases, PMSF was omitted from the lysis buffer. After centrifugation by microcentrifuge at 1,000×g for 10 min, the supernatant was stored in aliquots at −80° C.

ELISA

Ninety-six-well plates (Costar, N.Y.) were coated with affinity-purified capture MAbs (0.5 μg/well in 100 μl) at room temperature for 2 to 3 h. The coated plates were blocked with 3% bovine serum albumin (Sigma, Mo.) in PBS overnight at 4° C. Different recombinant PrP protein or 100 μl of diluted brain lysates (containing 60 μg of total brain proteins from a 20% total brain homogenate) was added to the wells. Plates were incubated at room temperature for 2 h and then washed with PBST (PBS with 0.05% Tween 20) three times before adding specific detecting biotinylated MAbs. After three additional washes with PBST, a horseradish peroxidase-streptavidin conjugate (Chemicon, Calif.) was added to the plates and incubated for 1 h. The plates were washed three times with PBST, and 100 μl of 2,2′-azinobis-(3-ethylbenzothiazoline-6-sulfonic acid) diammonium salt (Roche Diagnostic, Ind.) was dispensed into each well. After 15 min, the absorbance was read at 405 nm on a kinetic microplate reader (Molecular Device, Calif.). The results presented are the averages of the duplicates, and all experiments were repeated at least three times.

Enzymatic Treatment of Brain Homogenates.

Each brain homogenate was treated with 50 μg/ml of proteinase K (Sigma, Mo.) at 37° C. for 1 h. The protease was inactivated by the addition of PMSF to a final concentration of 3 mM.

SDS-PAGE and Immunoblotting.

Similar amounts of recombinant murine, human, bovine, or ovine PrP proteins were dissolved in 2× sample buffer and heated at 95° C. for 5 min before separation on 12% sodium dodecyl sulfatepolyacrylamide gel electrophoresis (SDS-PAGE). For reducing conditions, 2-mercaptoethanol (5% final concentration; Sigma) was added to the sample buffer. The polyacrylamide gel was transferred onto nitrocellulose membrane and probed by MAb 8H4 or MAb 7A12. After incubation with horseradish peroxidase-conjugated goat anti-mouse immunoglobulin G Fc (Chemicon, Calif.), the transferred PrP species were visualized using the chemiluminescence blotting system (Roche Diagnostics, N.J.).

Sucrose Gradient Fractionation.

Twenty percent total brain homogenate was prepared as described above, and Sarkosyl was added to a final concentration of 1%. After incubation for 30 minutes on ice, 0.5 ml of homogenate was loaded on a 10-to-60% step sucrose gradient in ultraclear centrifuge tubes (13 by 51 mm). Ultracentrifugation was carried out in SW55 rotors (Beckman, Calif.) at 200,000×g, 4° C., for 60 min. Fractions of 0.42 ml were collected from the top of the tube. In some experiments, blue dextran (molecular mass, 2,000 kDa; Sigma, Mo.) was included as a marker. To detect PrP species present in different sucrose gradient fractions by immunoblotting, 10 μl of each fraction was mixed with an equal volume of 2×SDS-PAGE buffer and heated at 95° C. for 5 min before being loaded onto a 12% gel, and immunoblot assays were carried out as described earlier. For aggregation-specific ELISA, 5 μl of each fraction in 95 μl of PBS was added to the ELISA plate using protocols described earlier.

Results

Rationale for the Development of an Aggregation-Specific ELISA.

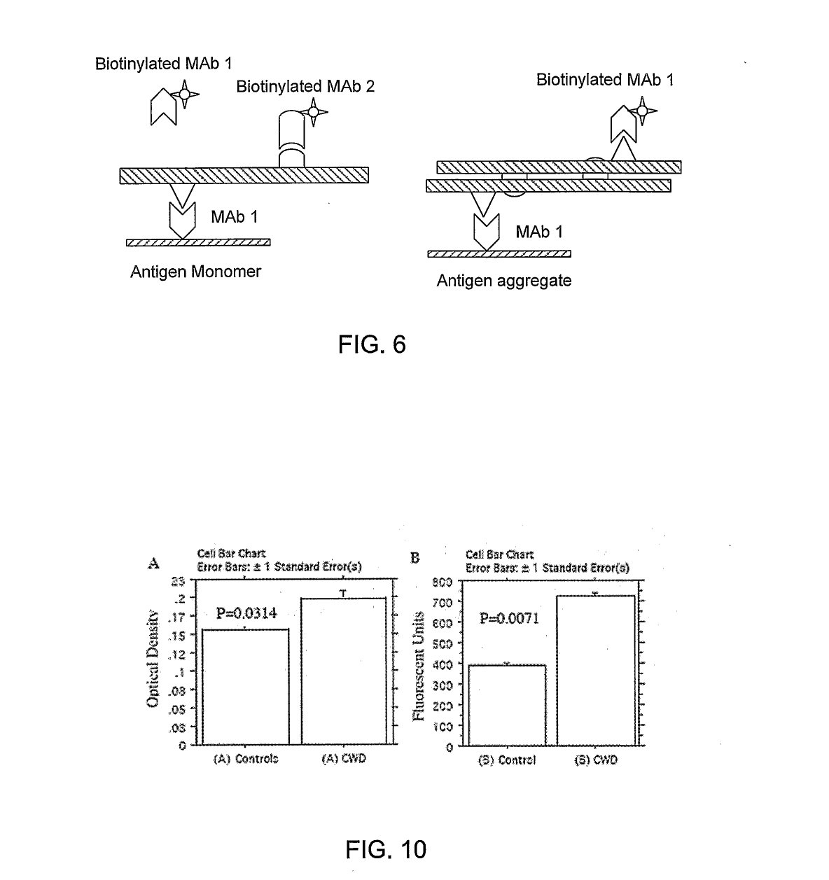

In a conventional sandwich or capture ELISA, two MAbs with distinct binding epitopes are required: one MAb is immobilized on a solid phase to capture the antigen, and a second MAb that reacts with a distinct epitope is then used to detect the bound antigen. We reasoned that when PrP protein dimerizes or aggregates, some MAb binding epitopes would be buried, while other MAb binding epitopes might be present more than once (FIG. 6). Accordingly, the number of epitopes available for binding will depend on the composition of the aggregate. If the PrP aggregate is a tetramer, then some MAb binding epitopes may be represented four times. We further postulated that if the epitope were present more than once, we might be able to use the same MAb for capture as well as detecting MAb. In addition, since dimers and aggregates are physically larger with repeating epitopes, they will have a stronger avidity with higher probability of being captured by the immobilized antibody, compared to a smaller PrP monomer. Therefore, in our dimer or aggregation-specific ELISA, one MAb is used as the capture MAb as well as the detecting MAb.

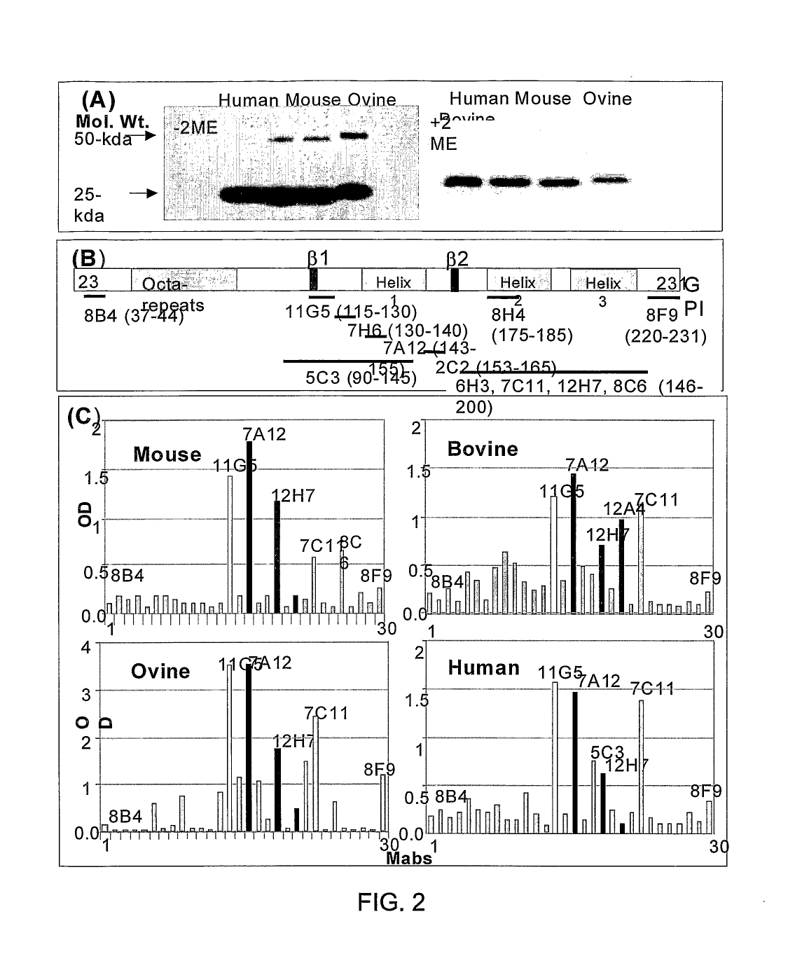

Whether all recombinant PrP (rPrP) proteins contain dimer is unresolved. We first confirmed that PrP dimer is present in rHu-PrP, rMo-PrP, rOv-PrP, and rBo-PrP proteins by immunoblotting. Using anti-PrP MAb 7A12 for detection, rHu-PrP, rMo-PrP, and rOv-PrP migrate as a 24- to 25-kDa protein with a small amount of dimeric PrP migrating as a 50-kDa protein (FIG. 2A, left panel). On the other hand, rBo-PrP migrates slower due to the presence of an additional octapeptide repeat. All dimeric rPrP contains a disulfide bond, because under reducing conditions only the 24- to 25-kDa monomeric rPrP is detected (FIG. 2A, right panel). Based on densitometry of the bands, the amount of dimeric rPrP is usually less than 5%. MAb 7A12 did not detect any rPrP with a molecular mass larger than a dimer.

Subsequently, we determined whether any of our 30 anti-PrP MAbs could react specifically with rMo-PrP dimer. The locales of some of the MAb binding epitopes are diagrammatically presented in FIG. 2B. Some MAbs react with linear sequences, while others react with conformational epitopes. Only five MAbs, 11G5 (amino acids [aa] 114 to 130), 7A12 (aa 143 to 155), 12H7 (conformational epitope from aa 145 to 200), 7C₁, and 8C6 (conformational epitopes from aa 145 to 200) consistently reacted strongly with rMo-PrP (FIG. 2C). Interestingly, neither the N-terminal-specific MAb 8B4 (aa 37 to 44) nor the C-terminal-specific MAb 8F9 (aa 220 to 231) detected any dimeric rMo-PrP; therefore, both the N terminus and the C terminus are not available for binding in the rMo-PrP dimers.

Four of these MAbs, 11G5, 7A12, 12H7, and 7C11, also reacted with rBo-PrP (FIG. 2C), rOv-PrP (FIG. 2C), and rHu-PrP (FIG. 2C) to various degrees. These results suggest that these recombinant PrP dimers share certain common features that are most noticeable in the central region, which contains the MAb 11G5 (aa 114 to 130) and 7A12 (aa 143 to 155) binding epitopes. However, the availability of some of these antibody binding epitopes is species specific. For example, MAb 8C6 reacted preferentially with rMo-PrP, MAb 12A4 reacted only with rBo-PrP, MAb 8F9 reacted most strongly with rOv-PrP, and MAb 5C3 reacted more strongly with rHu-PrP. Overall, the immunoreactivity with rOv-PrP was consistently higher.

The Aggregation-Specific ELISA is Specific for Dimeric PrP.

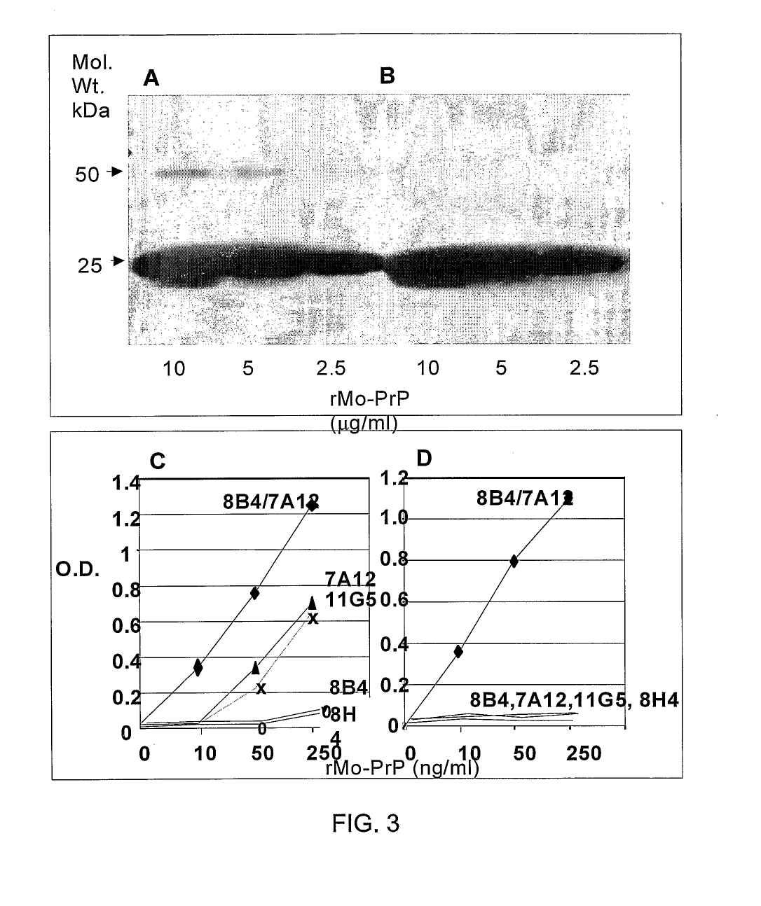

Aggregation of rMo-PrP is age dependent. Freshly prepared rMo-PrP does not contain PrP dimer. We prepared rMo-PrP proteins that are free of detectable dimeric PrP and rMo-PrP that contains dimer as described previously. The presence of dimeric rMo-PrP in the preparation was first confirmed by immunoblotting with MAb 7A12. It was clear that dimeric rMo-PrP was present only in sample A (FIG. 3A) and not in sample B (FIG. 3B). We also used a conventional capture ELISA using MAb 8B4 as the immobilized, capture MAb and biotinylated MAb 7A12 as the detecting MAb to verify that the two preparations of rMo-PrP proteins had comparable amounts of protein (FIGS. 3C and 3D).

We next determined whether the MAbs identified earlier indeed react only with dimeric rMo-PrP. When tested in the dimer-specific ELISA, MAbs 7A12 and 11G5, but not 8H4 or 8B4 reacted strongly with the rMo-PrP preparation, sample A that contains dimeric rMo-PrP, in an antigen concentration-dependent manner (FIG. 2C). On the other hand, none of the tested MAbs (11G5, 7A12, 8H4, or 8B4) was able to react with sample B, which lacks rMo-PrP dimer (FIG. 3D). Furthermore, the dimer-specific ELISA is about 100-fold more sensitive than the immunoblot assays. These results provide strong evidence that our aggregation-specific ELISA indeed reacts with rMo-PrP dimers.

Detection of PrP Aggregates in PrP^{Sc}-Infected Mouse Brains.

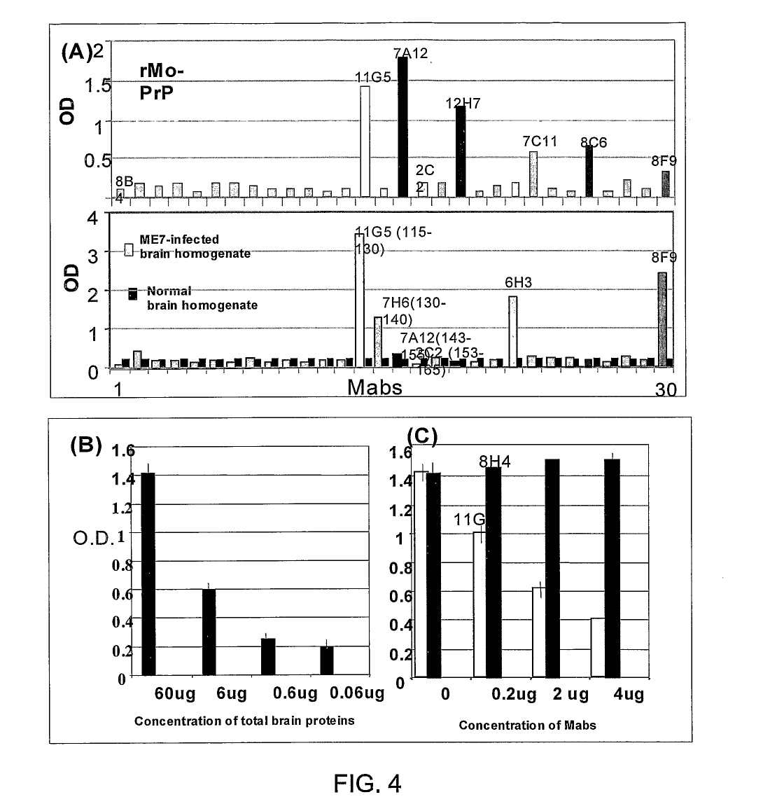

We next determined whether the aggregation-specific ELISA could detect PrP aggregates present in brain homogenates from mice infected with the ME7 strain of PrP^{Sc}. We screened the 30 anti-PrP MAbs and compared the immunoreactivities detected in infected brains with normal sham-infected control brains. Brain homogenates from Prnp^{-/-}-129/Ola mice were also used as negative controls. To assist the comparison, the results from rMo-PrP are also presented (FIG. 4A). None of the anti-PrP MAbs had significant immunoreactivity with brain homogenates from either sham-infected control mice or Prnp^{-/-}-129/Ola mice (not shown). Only 4 of the 30 anti-PrP MAbs reacted strongly with brain homogenates from infected mice (FIG. 4B).

Interestingly, MAbs 8C6, 7A12, and 12H7, which reacted strongly with rMo-PrP protein, did not react with infected brain homogenates at all. On the other hand, MAbs 7H6, 6H3, and 8F9, which did not react with rMo-PrP, reacted robustly with infected brain homogenates. Only MAb 11G5 reacted with both rMo-PrP proteins and infected brain homogenates. Furthermore, the immunoreactivity differences between infected and normal brain homogenates were profound. For example, when tested with MAb 11G5, the difference in immunoreactivity (as defined by the optical density [OD]) between infected and normal brain homogenate was more than 300%. The immunoreactivity detected with MAb 11G5 was also much stronger in infected brains than in the rMo-PrP preparation. Overall, these results suggest that the nature of the PrP aggregates formed in infected brain and rMo-PrP dimer are different.

By using MAb 11G5, we found that the aggregation-specific ELISA could detect signals in an infected brain homogenate, which contains between approximately 0.6 and 6 μg of total brain proteins (FIG. 4C, left panel). Furthermore, the binding of biotinylated 11G5 was blocked by unconjugated MAb 11G5 in a MAb-specific and concentration-dependent manner. The irrelevant MAb 8B4 did not block (FIG. 4C, right panel). When ELISA plates were first coated with an irrelevant, non-anti-PrP MAb, there was no binding of biotinylated MAb 11G5 (not shown). Therefore, the aggregation-specific ELISA is both antigen and antibody specific.

The Aggregation-Specific ELISA is Applicable to Two Other Strains of PrP^{Sc}.

We next determined whether our findings in ME7-infected brains are applicable to two other strains of PrP^{Sc}, 22L and 139A. Individual brain homogenate was prepared from ME7-infected (n=4), 22L-infected (n=4), and 139A-infected (n=4) mice at the terminal stages of disease, and each homogenate was analyzed individually. We found that MAb 11G5/11G5 also reacted with PrP aggregates in mice infected with either 22L or 139A PrP^{Sc} (Table 1). Similar to ME7-infected brains, the immunoreactivity was robust and highly reproducible. Thus, the aggregate-specific assay is applicable to at least three mouse PrP^{Sc} strains.

The Immunoreactivity Detected by the Aggregation-Specific ELISA is Associated with PK-Resistant PrP Species.

One cardinal feature of PrP^{Sc} is its PK resistance. We determined whether the binding activity detected with MAb 11G5/11G5 is associated with PK-resistant PrP species in mice infected with ME7 (n=4), 139A (n=4), or 22L (n=4) PrP^{Sc}. Each brain homogenate was divided into two tubes. One was treated with PBS, and the other was treated with 50 μg/ml of PK as described earlier. PK digestion did not reduce the binding in 22L-, 139A-, or ME7-infected brains (Table 1). Therefore, the PrP aggregates detected in this assay are PK resistant and most likely represent PrP^{Sc}.

The Aggregation-Specific ELISA Reacts with PrP^{Sc} Aggregates of Various Sizes in Infected Brains.

Recently, we found that after ultracentrifugation in a 10-to-60% sucrose gradient, all the mouse PrP^{C} species are present in the top fractions, mainly in fractions 1 and 2. In sharp contrast, in PrP^{Sc}-infected mouse brain, immunoreactivity is present in all fractions, with the strongest reactivity present in the bottom fractions, fractions 10 and 11. These bottom fractions are known to contain the largest PrP^{Sc} aggregates.

TABLE 1
Detection of PrP aggregates in brain homogenates from mice infected with
one of the three different strains of PrP^{Sc} and PK resistance of aggregates^{a}
OD of homogenates from mice infected with:

| Treatment | Control (n = 4) | ME7 (n = 4) | 139A (n = 4) | 22L (n = 4) |
|---|---|---|---|---|
| PBS | 0.303 +/− 0.02 | 2.41 +/ 0.12^{b} | 2.18 +/− 0.07^{c} | 3.10 +/− 0.08^{d} |
| PK | 0.190 +/− 0.003 | 2.52 +/− 0.01^{e} | 2.30 +/− 0.04^{f} | 2.98 +/− 0.12^{g} |
| ^{a}Individual brain homogenate was first treated with PBS or PK, as described, prior to the ELISA. Aggregation-specific ELISA was carried out as described in FIG. 6 using MAb 11G5/11G5. Results presented are the mean ± the standard error of the OD of four individual mice. | | | | |
| # P values were determined by paired Student's t tests. All infected brains had munch stronger immunoreactivity than control normal brains, and the immunoreactivity detected with the aggregation-specific ELISA was still present after treatment of the brain homogenates with PK. | | | | |

^{b}For PBS-treated ME7 infected brain homogenates versus PBS-treated control brain homogenates, P = 0.0003.
^{c}For PBS-treated 139A-infected brain homogenates versus PBS-treated control-brain homogenates, P = 0.0002.
^{d}For PBS-treated 22L-infected brain homogenates versus PBS-treated control-brain homogenates, P = 0.0001.
^{e}For PK-treated ME7-infected brain homogenates versus PK-treated normal control homogenates, P < 0.0001.
^{f}For PK-treated 139A-infected brain homogenates versus PK-treated normal control homogenates, P < 0.0001.
^{g}For PK-treated 22L-infected brain homogenates versus PK-treated normal control homogenates, P < 0.0001.

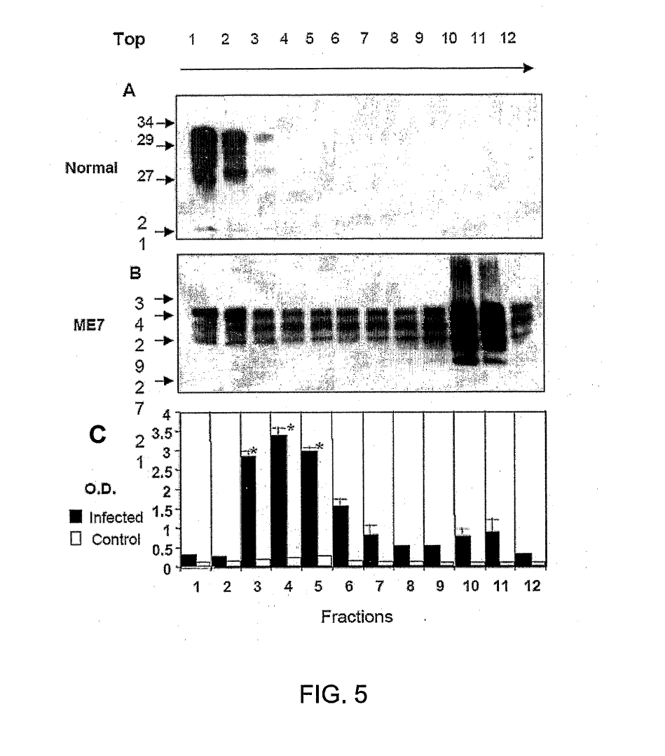

We fractionated one control brain homogenate and one ME7-infected brain homogenate in a sucrose gradient. Each fraction was then collected and divided into two samples; one sample was immunoblotted with MAb 8H4, which detects all PrP species, to document the distribution of the PrP species, and the second sample was used to detect PrP aggregate using MAb 11G5 in the aggregation-specific ELISA. Consistent with our earlier findings, in control mice, all PrP^{C} binding activity was present in fractions 1 and 2 (FIG. 5A). In contrast, the PrP immunoreactivity in infected brain was dispersed among all fractions, with fractions 10 and 11 having the highest intensities (FIG. 5B).

We did not detect any MAb 11G5 immunoreactivity in any of the fractions from normal control mice (FIG. 5C). In contrast, MAb 11G5 detected high levels of immunoreactivity in fractions 3, 4, and 5. On the other hand, fractions 10 and 11, which have the largest PrP aggregates, also contained MAb 11G5 immunoreactivity, but the levels were much lower (FIG. 5C). Blue dextran has a molecular mass of about 2,000 kDa, and it partitions in fraction 3 when centrifuged under identical conditions. Therefore, a majority of the PrP^{Sc} aggregates detected by this assay are PrP aggregates of heterogeneous size with molecular mass ranging from around 2,000 kDa to larger than 2,000 kDa, but these aggregates are smaller than the largest PrP^{Sc} aggregates present in fractions 10 and 11. This experiment has been repeated with three additional ME7-infected brain homogenates as well as brain homogenates from 139A-infected or 22L-infected mice with comparable results (not shown).

Detection of PrP Aggregates During Disease Progression.

ME7-inoculated CD-1 mice begin to show signs at about 130 to 160 days postinfection and die within 3 weeks. Previously, we found that PK-resistant PrP species are only detected in animals infected 140 days earlier. To determine when the PrP aggregates first become detectable, brain tissues were obtained from sham-infected mice (n=4), mice infected 30 days (n=4) or 70 days earlier (n=4) and not exhibiting clinical signs, mice infected 140 days earlier (n=4) and exhibiting obvious clinical signs, and mice at a terminal stage (about 170 days) of disease (n=4). Significant PrP aggregate immunoreactivity was detected in animals 70 days postinfection, at a time when PK-resistant PRP species are undetectable by immunoblotting (Table 2). These results suggest PrP aggregates are detectable in infected animals much earlier than the manifestation of clinical signs and the detection of PK-resistant PrP species.

TABLE 2
Appearance of PrP aggregates during disease progression in ME7-infected
mice^{a}

| | Days postinfection | | | | |
|---|---|---|---|---|---|
| Parameter | Control (n = 4) | 30 (n = 4) | 70 (n = 4) | 140 (n = 4) | 170 (n = 4) |
| OD | 0.56 +/0.11 | 0.55 +/− 0.03 | 0.76 +/− 0.05 | 2.65 +/− 0.12 | 2.85 +/− 0.13 |
| P value^{b} | | 0.65 | 0.035 | 0.004 | 0.006 |

^{a}Brain lysates were prepared from individual control mice or mice infected with ME7 PrP^{Sc} at 30, 70, 140, 94 170 days earlier. Aggregation-specific ELISA was carried out as described in FIG. 5 with MAb 11G5/11G5. Results were the means for the four micey ± the standard error. P values were determined by paired Student's t tests.
^{b}Comparing infected brain homogenates from each time point to normal control brain homogenates.

Discussion

A panel of 30 different MAbs were developed against recombinant PRP, and by screening these anti-PrP MAbs, we have identified five MAbs that preferentially react with rMo-PrP dimers in a dimer-specific ELISA (FIG. 6). Most noteworthy are MAbs 11G5 (aa 114 to 130) and 7A12 (aa 143 to 155), which also react strongly with rBo-PrP, rOv-PrP, and rHu-PrP. Therefore, the epitopes recognized by these two MAbs are conserved across these four species. On the other hand, the binding of the other three MAbs is more variable between rPrP from different species. We also identified MAbs that are species specific, which may reflect the conformational differences among the recombinant PrP or PrP dimers from these four animal species.

None of the 30 MAbs has significant binding with brain homogenates from control, sham-infected mice or Prnp^{-/-} mice. Therefore, if dimeric PrP^{C} is present in normal brain, it is not detected with these MAbs. Of the five MAbs that reacted with rMo-PrP dimers, only MAb 11G5 reacted strongly with brain homogenates from ME7-infected mice. On the other hand, MAb 7A12, which reacts strongly with all four tested recombinant PrP dimers, did not react with infected mouse brain homogenates. This result suggests that as a consequence of PrPc-to-PrPsc conversion the helix 1 region (aa 143 to 157) of the molecule has changed. The helix 1 region may be important in the pathogenesis of prion disease. In a cell model, helix 1 of PrP is a major determinant of PrP folding. Disruption of helix 1 prevents the attachment of the glycophosphatidylinositol anchor and the formation of the N-linked glycans. In the absence of the glycophosphatidylinositol anchor, helix 1 induces the formation of unglycosylated and partially protease resistant PrP aggregates. In all sequenced PrP^{C} with the exception of rodent PrP^{C}, this region also contains the sequence DYEDRYYREN, which is composed entirely of hydrophilic amino acids. In rodents PrP^{C}, the second amino acid, tyrosine (Y), is replaced with a tryptophan. It has been suggested that this region is important in the formation of the hydrophilic core and seeding of PrP aggregates. This region also contains the YYR epitope, which has been reported to be exposed only in PrP^{Sc} and is not available for binding in PrP^{C}. Biophysical studies have also provided strong evidence that PrPc-to-PrPsc conversion involves the conversion of α-helix 1 to a β-sheet structure. Another explanation for the inability of 7A12 to detect native PrP^{Sc} aggregate may be due to the presence of N-linked glycans. The presence of N-linked glycan in PrP^{Sc} aggregates may interfere with the binding of MAb 7A12.

On the other hand, MAb 11G5 reacts with both rPrP dimers and PrP^{Sc} aggregates in infected brains. The epitope of MAb 11G5 (aa 114 to 130) includes the first β-strand (aa 128 to 131). Hence, the conformation of this region may be similar between rPrP dimer and PrP_{Sc} aggregates, and PrPc-to-PrPsc conversion may not change the overall conformation of this region. In vitro studies using synthetic peptide have also identified residues 119 to 136 on PrP^{C} to be important in the conversion process. Analysis of 27 mammalian and 9 avian PrP proteins revealed that the most conserved region outside the globular domain is located between residues 113 and 137; thus, this part of the molecule must be important in the biology of PrP^{C}.

Worthy of note is that the binding of MAb 11G5 to infected brain homogenate is about 300% higher than to rMo-PrP. Since rMo-PrP dimer has two MAb 11G5-reactive epitopes, the PrP aggregates present in infected brain may contain multiple PrP molecules with more than two MAb 11G5-reactive epitopes. Furthermore, MAb 11G5 also reacts with brain homogenates from animals infected with either one of the two other strains of mouse PrP^{Sc}, namely, 139A and 22L. Hence, while different strains of PrP^{Sc} are known to have different conformations, the MAb 11G5 epitope on PrP^{Sc} aggregate is shared between three different strains of mouse PrP^{Sc}. We also identified three MAbs, 7H6, 6H3, and 8F9, which did not react with rMo-PrP dimer but reacted significantly with infected brain homogenates. The MAb 7H6-reactive epitope (aa 130 to 140) is contiguous to the MAb 11G5-reactive epitope and right before the helix 1 region. MAb 6H3 reacts with a conformational epitope which is located at the C-terminal region. The MAb 6H3-reactive epitope is quite unusual, as its availability for binding is critically dependent on the N terminus. Previously, we reported that binding of MAb 6H3 to recombinant rHu-PrP could be blocked by the binding of MAb 8B4, which binds to the N-terminal end of rHu-PrP. Accordingly, we speculated that there might be interactions between the N terminus and C terminus of the rHu-PrP protein. However, the relationship between these observations and the presence of multiple MAb 6H3-reactive epitopes in PrP^{Sc} aggregates in infected brains is not clear.

MAb 8F9 (aa 220 to 231) does not react with rMo-PrP dimer in our dimer-specific ELISA but reacts significantly with the PrP^{Sc} aggregates in infected brains. Recently, a MAb was generated by immunizing mice with a linear sequence encompassing residues from 214 to 226 of PrP. This MAb reacts with a conformational epitope which is available for binding in PrP^{Sc} but not in PrP^{C} or recombinant PrP. Both of these results suggest that the conformation of the C terminus is amenable to change during the conversion process.

By ultracentrifugation in a sucrose gradient, most of the immunoreactivity detected by the aggregation ELISA is present in fractions 3, 4, and 5. These fractions are mostly devoid of PrP^{C}, as it partitions to the top two fractions. Interestingly, while the bottom fractions also have immunoreactivity, the levels of binding are much lower in these largest PrP^{Sc} aggregates, which indicates that this aggregate-specific ELISA is more efficient in detecting smaller aggregates than the larger one. When fractionated in the same gradient, the 2,000-kDa blue dextran is present in fraction 3. Therefore, the aggregated PrP^{Sc} being detected is likely to have a mass similar to blue dextran and larger.

At the present time, the relationship between the PrP aggregates detected by the aggregation-specific ELISA and the largest PrP^{Sc} aggregates in the infected brains is not known. Using an in vitro conversion assay, it is estimated that the “seed” or converting activity associated with PrP^{Sc} is heterogeneous in size but larger than the molecular mass standard, blue dextran. It should be noted that while all fractions from sham-infected mice are sensitive to PK digestion, PK-resistant PrP species are detected in all fractions from infected mice (results not shown). These results suggest that the size of PK-resistant PrP species is heterogeneous. Hence, the aggregation-specific ELISA detects only a subpopulation of the PK-resistant species. The composition of these PrP^{Sc} aggregates is not known. In addition to PrP, it is possible that they may contain other cellular components, such as nucleic acids, lipids, non-PrP proteins, polysaccharides, or glycosaminoglycans.

Our time-course studies in ME7-infected mice revealed that the appearance of the PK-resistant PrP species and clinical signs all occur around the same time, at 140 days post-inoculation. On the other hand, using MAb 11G5 the accumulation of the PrP^{Sc} aggregate was detected earlier, at 70 days post-infection. We did not detect any immunoreactivity in animals infected 30 days earlier. These results are in agreement with our recent finding that by using an epitope-scanning assay to detect changes in the conformations of PrP^{Sc}, the earliest change detected was in animals infected approximately 70 days earlier. We also found that, at this time, aberrant full-length and truncated PrP species begin to accumulate in the infected brains. The PrP^{Sc} we are detecting with the aggregation ELISA is PK resistant. The reason that we can detect PrP^{Sc} at 70 days post-inoculation is most likely because the ELISA is more sensitive than immunoblot assays. The assay can routinely detect significant binding in lysates containing between 0.6 and 6 μg of total brain proteins. Using rMo-PrP as standard, we estimated that the PrP^{C} in normal brain accounts for approximately 0.01% of the total brain proteins (results not shown). Therefore, the aggregation ELISA has the potential to detect between 0.06 and 0.006 μg of aggregated PrP. However, it will require highly purified PrP^{Sc} aggregates to precisely determine the sensitivity of the aggregation-specific ELISA. So far, the accumulation of PrP aggregates during disease progression has only been carried out with MAb 11G5. MAb 11G5 is specific for an epitope (aa 115 to 130) at the central region, and this region is exposed in the recombinant PrP dimeric structure. The majority of the screened anti-PrP MAbs cannot react with the PrP^{Sc} aggregates at the terminal stage of disease, which may be caused by the masking of their binding sites during the progressive aggregation of PrP^{Sc}. Therefore, it is possible that by using other MAbs we may be able to detect PrP aggregates at earlier time points after infection, when PrP aggregates are smaller.

Currently, all the in vitro diagnostic tests for prion diseases require either the demonstration of PK-resistant PrP species in brain homogenate or the uncovering of hidden epitopes after GdHC1 treatment. The aggregation-specific ELISA described here provides certain advantages: (i) the assay is much more sensitive because of the use of a capture antibody; (ii) the assay is simpler because a denaturing step is not required; and (iii) with the use of more than one antibody, a built-in control can be used to monitor the specificity and sensitivity of the assay. While all the tests were carried out in infected mice, based on our studies with rOv-PrP, rBo-PrP, and rHu-PrP, it is likely that the same approach can be used to develop a test for other animal prion diseases as well as in human prion diseases.

Indeed, recent results suggest that the aggregate-specific assay also detects PrP^{Sc} in Creutzfeldt-Jacob disease patients. Finally, the principle of the aggregate-specific ELISA we have developed may be applicable to other diseases caused by abnormal protein aggregation, such as Alzheimer's disease or Parkinson's disease.

### Example 2

### An Ultra Sensitive Assay for Prion Detection

Introduction

We developed a novel aggregation specific ELISA (AS-ELISA) that is specific for PrP aggregate. In a conventional sandwich-ELISA, two monoclonal antibodies (MAbs) with distinct binding epitopes are required. We reasoned that when PrP protein dimerizes or aggregates, some MAb binding epitopes would be buried, while other MAb binding epitopes might be present more than once. Therefore, in AS-ELISA, one MAb is used as the capture-MAb as well as the detecting-MAb. The assay can detect PrP^{Sc} aggregates in the brain of mice 70 days post-intracerebral inoculation, at a time when no PK-resistant PrP is detectable.

Fluorescent Amplification Catalyzed by T7 RNA polymerase Technique (FACTT) is another newly developed assay. In this system, a biotin-labeled amplification module (AM), T7 promoter, is coupled directly to streptavidin that also binds to biotinylated detection MAbs. The amplification is triggered in an isothermal and linear manner using T7 RNA polymerase, the product is then detected by a fluorescent dye. Such a system can detect protein targets at sub-femtomolar levels. We describe the further improvement of our assay by combining AS-ELISA with FACTT to develop a new assay, AS-FACTT. The AS-FACCT is at least 1,000 folds more sensitive than AS-ELISA in detecting recombinant PrP dimers. Furthermore, we describe the use of AS-FACT to follow the temporal appearance of PrP^{Sc} aggregates in the brain of mice inoculated with infectious prion peripherally. Finally, we show that the principle of AS-FACT is applicable to deer and elk with CWD and human with vCJD.

Materials and Methods

Recombinant PrP Protein and Monoclonal Antibodies

The generation of human, mouse, ovine and bovine recombinant PrP proteins and anti-PrP MAbs have been described in detail. All MAbs were affinity purified with Protein G chromatography. MAbs were biotinylated using the EZ-Link™ Sulfo-NHS-Biotin kit (Pierce Endogen, Rockford, Ill.).

Mice

ME7 or 139A mouse-adapted scrapie strains were injected (0.1 ml of a 10% brain homogeneity) by intraperitoneal (i.e.) injection into 7-week-old CD-1 mice as previously described. ME7 has a titer of 10⁸ID₅₀/ml and 139A has a titer of about 10⁷ID₅₀/ml. Sham-infected, age-and sex-matched, CD-1 mice as well as PrP^{C} knock out mice were used as controls. All animal experiments were carried out according to institutional regulations and standards.

Human Tissues

For controls, brain autopsies from the frontal cortex were obtained from individuals (n=8) confirmed negative for prion disease. for vCJD (n=8) and sCJD (n=8), frontal cortexes were obtained from cases confirmed at autopsy. All samples from vCJD and sCJD cases have proteinase K-resistant PrP^{Sc}, as demonstrated by immunoblotting with anti-PrP MAb 8H4 (not shown).

Deer and Elk Tissues

Brain tissues from deer and elks with histological-confirmed CWD (n=3) and non-CWD controls (n=6) were kindly provided by Dr. Elizabeth S. Williams of Wyoming State Veterinary Laboratory, University of Wyoming, Laramie, Wyo. and Dr. Michael Miller of Division of Wild Life, Fort Colin, Colo. All CWD Animals have proteinase K-resistant PrP^{Sc} as demonstrated by immunoblotting with anti-PrP MAb 8H4 (not shown).

Preparation of Brain Homogenate

To prepare 20% (w/v) total brain homogenate, individual entire brain samples were homogenized in ice-cold lysis buffer (phosphate-buffered saline, with 1% Nonidet P-40, 0.5% sodium deoxycholate, 5 mM EDTA, pH 8.0) in the presence of 1 mM phenylmethylsulfonyl fluoride (PMSF) (Sigma, Mo.) as described. If the homogenate was to be treated with proteases, PMSF was omitted from the lysis buffer. After centrifugation by micro-centrifuge at 1000 g for 10 minutes, the supernatant was stored in aliquots at −80° C.

AS-ELISA Assay

Ninety-six-well plates (Costar, N.Y.) were coated with affinity-purified capture MAbs (0.5 μg/well in 100 μl) at room temperature for 2-3 hrs. The coated plates were blocked with 3% Bovine Serum Albumin (Sigma, Mo.) in PBS overnight at 4° C. Different recombinant PrP protein (ng/ml) or one hundred microliters of diluted brain lysates (containing 60 μg of total brain proteins from a 20% total brain homogenate) were added to the wells. Plates were incubated at room temperature for 2 hrs and then washed with PBST (PBS with 0.05% Tween-20) three times before adding specific detecting biotinylated MAbs. After three additional washes with PBST, a HRP (horseradish peroxidase) strepavidin conjugate (Chemicon, Calif.) was added to the plates and incubated for 1 hour. The plates were washed three times with PBST, and 100 μl of ABTS (2,2′-azinobis-[3-ethylbenzothiazoline-6-sulfonic acid] diammonium salt) (Roche Diagnostic, IN) was dispensed into each well. After 15 minutes, the absorbance was read at 405 nm on a Kinetic Micro-plate Reader (Molecular Device, Calif.). The results presented were the average of the duplicates, and all experiments were repeated at least three times.

AS-FACT Assay

For AS-FACT assay, the capture antibody was coated in carbonate-bicarbonate buffer (pH9.6) to a 384-well plate at 5 μg/ml and 20 μl/well for overnight at 4° C. The plate was washed 3 times by PBST (0.1% Tween-20 in PBS), and blocked with 1% casein in PBST for 1 hr. After 3 times wash with PBST, the tested samples (from a 20% total brain homogenate) were diluted in PBS and added into the coated plate in the amount of 20 μl per well, for a 60 min incubation at room temperature. The plate was washed 3 times with PBST, and 20 μl of a diluted biotinylated detection antibody (1 μg/ml) was added in each well. Plate was incubated at room temperature for 30 min. Streptavidin and biotin-DNA template (the amplification module, AM) were added sequentially at 5 μg/ml and 250 μg/ml respectively, with 30 min room temperature incubation for each step, followed by three times wash with PBST between each binding incubation. After excess AM and proteins were removed by washing, 20 μl of reaction mixture, which contains 60 units of T7 RNA polymerase plus (Ambion), 1.25 μM NTP, 1× T7 buffer (Ambion) was added to each well. RNA amplification was performed at 37° C. for 3 hours. The RNA intercalating dye, RiboGreen (Molecular Probes) was added to the reaction mixture (20 μl, 1:200 diluted in the TE buffer supplied by the manufacturer) and the plate was read at Ex 485 nm/Em 535 nm in a TECAN SpectraFluor reader.

Statistical Analysis:

Paired Student's t test was used to calculate the statistical difference of experimental versus control values. Difference is considered statistically insignificant if P>0.05.

Results

AS-FACT is More Sensitive than AS-ELSA in Detecting Recombinant PrP (rPrP) Dimers

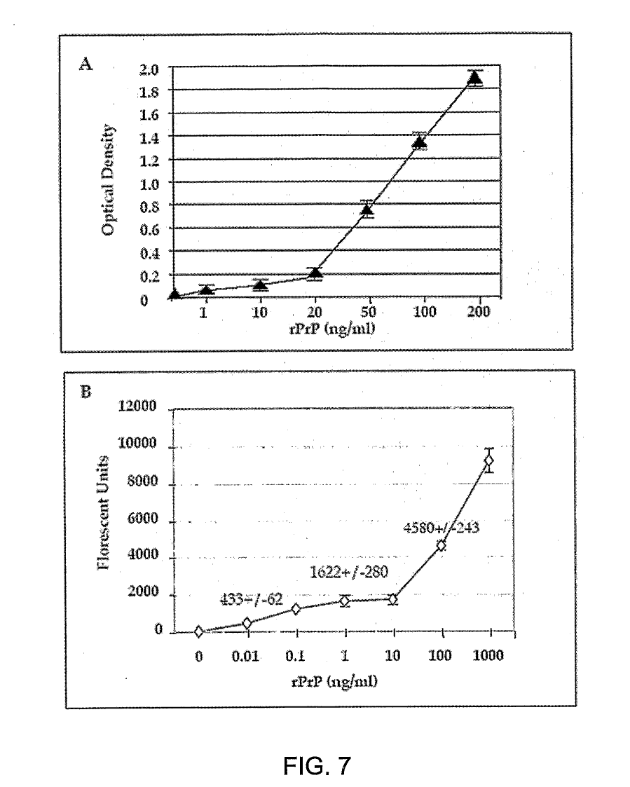

We first demonstrated that AS-FACT was more sensitive than AS-ELISA in detecting mouse rPrP dimers. Using MAb 11G5, the lowest detecting limit of AS-ELISA was at about 20 ng/ml or 2 ng/well of rPrP (FIG. 7A). On the other hand, AS-FACT could detect 10 pg/ml or 2 pg/well of rPrP (FIG. 7B). Since about 5% of recombinant rPrP is present in dimeric form, we estimate that AS-ELISA has a detection limit at about 100 pg of rPrP dimers, while AS-FACT has a detection limit at about 100 fg. In general, the AS-FACT is approximately 1,000 to 10,000 folds more sensitive than AS-ELISA in detecting rPrP dimers.

The AS-FACT is also More Sensitive in Detecting PrP^{Sc} Aggregates in PrP^{Sc} Infected Brains

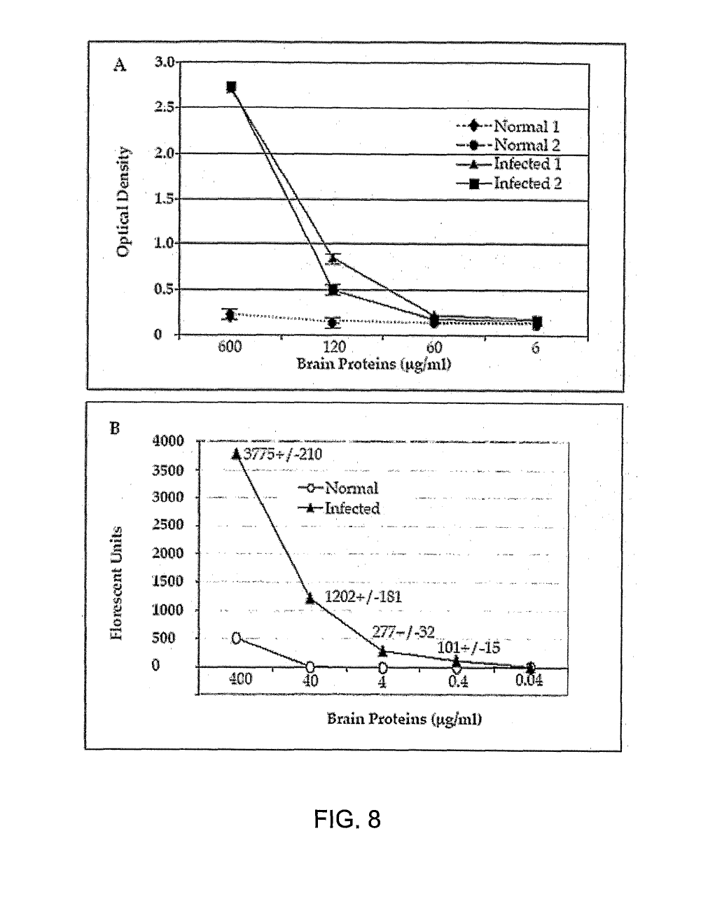

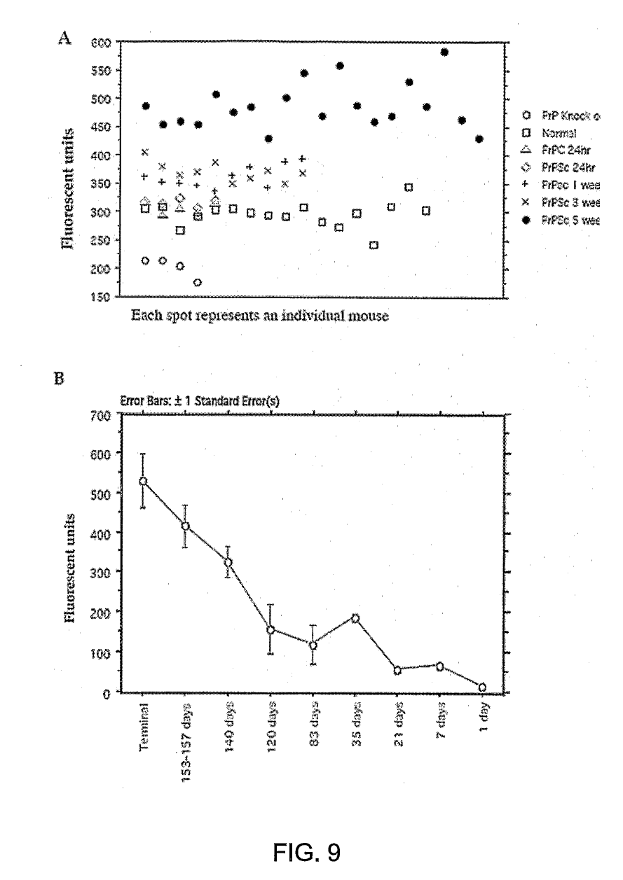

We next compared the sensitivity of AS-FACT and AS-ELISA in detecting PrP^{Sc} aggregates in the brains of mice infected with ME7 PrP^{Sc}. Brain homogenates from non-infected control or from infected, terminally sick mice were serially diluted and assayed for the presence of PrP^{Sc} aggregates by AS-ELISA or AS-FACT. In AS-ELISA, at 120 μg/ml of total brain proteins, the immunoreactivity detected in PrP^{Sc}-infected brain homogenates was significantly higher than the controls (FIG. 8A). However, when the protein concentration was diluted to 60 μg/ml the difference was not longer significant. On the other hand, AS-FACT was able to detect significant immunoreactivity in infected brains, even at 0.4 μg/ml of total brain proteins (FIG. 8B).

Detection of PrP^{Sc} Aggregates in the Brains of Animals Inoculated with PrP^{Sc} Intra-Peritoneally at Different Time Points

Previously, we found that AS-ELISA could first detect PrP^{Sc} aggregates at approximately 70 days but not at 30 days post inoculation of PrP^{Sc}. We next determined whether AS-FACT could detect PrP^{Sc} aggregates at a time when AS-ELISA was unable to do so. A group of mice was inoculated with ME7 PrP^{Sc} intraperitoneally. We inoculated animals intra-peritoneally rather than intracerebrally to avoid the possibility of detecting the injected PrP^{Sc}. At 35 days after inoculation, brain from individual mouse was prepared and assayed for the presence of PrP^{Sc} aggregates by either AS-ELISA or AS-FACT. Brains from a group of similarly infected mice, but at terminal stages of disease (>180 days post i.p. inoculation) were used as positive controls. Brains from non-infected control mice were used as negative controls. All terminally sick mice had PK resistant PrP^{Sc} species, while none of the animal infected 35 days earlier had any (results not shown). As expected, both AS-ELISA and AS-FACT detected PrP^{Sc} aggregates in every animal at terminal stages of disease (Table 3). However, in brains from animals inoculated intraperitoneally 35 days earlier, only AS-FACT was able to detect PrP^{Sc} aggregates; with 100% sensitivity and specificity.

TABLE 3

| Normal controls^{(a)} | | | 35 days post infection^{(b)} | | Terminally ill^{(c)} | |
|---|---|---|---|---|---|---|
| | AS- | | AS- | | AS- | |
| | ELISA | AS-FACT | ELISA | AS-FACT | ELISA | AS-FACT |
| | (n = 13) | (n = 17) | (n = 10) | (n = 20) | (n = 10) | (n = 20) |
| Mean +/− S.D. | 0.33/−0.07 | 295 +/− 21.42 | 0.356 =/− 0.05 | 487 +/− 41.44 | 1.95 +/− 0.55 | 841+/205 |
| Mini. | 0.200 | 243 | 0.290 | 429 | 1.34 | 630.2 |
| Max | | | 0.450 | 585 | 2.70 | 1200 |
| P value | | | 0.352* | <0.0001* | <0.0001* | <0.0001* |

^{(a)}Brain homogenates were prepared from individual, CD1 mouse. Each homogenate was assayed in AS-ELISA or AS-FACT as described in Material and Methods.
^{(b)}CD-1 mice were inoculated i.p. with 0.1 ml/mouse of a 10% brain homogenate from terminally sick CD-1 mice infected with ME7 PrP^{Sc}. At 35 days after inoculation, each brain was removed and homogenate prepared. Each homogenate was assayed in AS-ELISA or AS-FACT as described in Material and Methods.
^{(c)}Terminally ill mice were CD-1 mice infected >170 days earlier with ME7 or 139A PrP^{Sc} and showing signs of prion disease.
*Compare to non-infected control

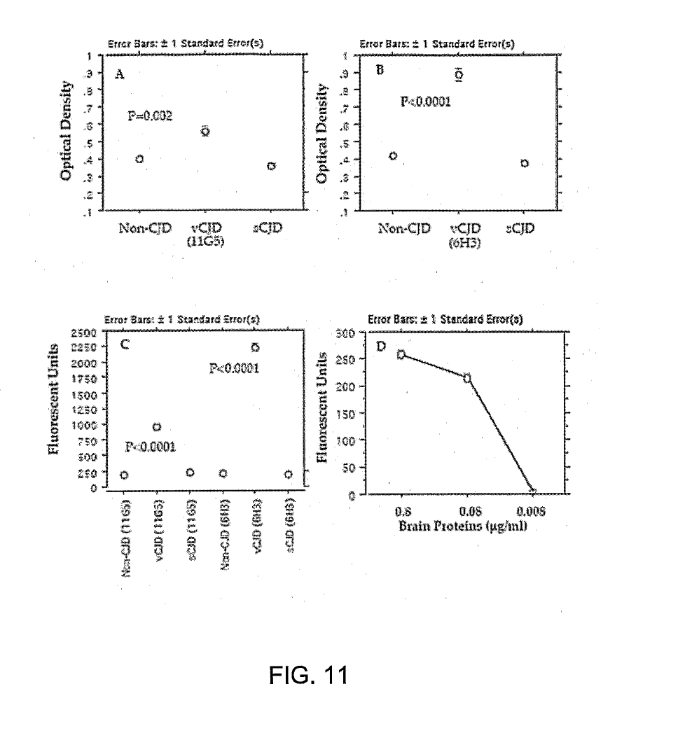

We next investigated the temporal appearance of PrP^{Sc} in the brains of mice inoculated intra-peritoneally. A group of animals was inoculated with PrP^{Sc} intraperitoneally. At 1 day (n=5), 7 days (n=10), or 21 days (n=10) after inoculation, brain homogenate from individual mouse was prepared. A group of animals (n=5) was inoculated with normal brains and their brain harvested 1 day later to serve as controls. Brains from PrP^{C-/-} mice (n=4) were also included as negative controls. Each brain homogenate was assayed for PrP^{Sc} aggregate by AS-FACT (FIG. 9A). The results from individual mouse inoculated 35 days earlier were included for comparison. The immunoreactivity detected in normal mice, mice injected with normal brain homogenates, or mice injected with PrP^{Sc} one day earlier were comparable. However, significant immunoreactivity was detected in 7 out of the 10 mice injected 7 days earlier with PrP^{Sc}. The levels of immunoreactivity increase slowly, as infection progresses. The results of additional experiments with more time points are summarized in FIG. 9B.

Detection of PrP^{Sc} Aggregates in Deer and Elk with Chronic Wasting Disease (CWD)

We next investigated whether the aggregation specific assay was applicable to prion diseases in other animals, such as deer and elk with CWD, a form of prion disease in cervidae. By AS-ELISA with MAb 11G5, all CWD brain homogenates (n=3) had slight but significantly higher immunoreactivity than the non-CWD controls (n=6) (FIG. 10A). As expected, the differences between CWD and non-CWD controls were significantly amplified by using MAb 11G5 in AS-FACT (FIG. 10B). Therefore, the principle of AS-FACT is applicable to CWD.

Detection of PrP^{Sc} Aggregates in vCJD

We also investigated whether the assays could detect human PrP^{Sc} aggregates in sCJD and vCJD. We detected a slight increase in MAb 11G5 immunoreactivity in all vCJD samples (n=8), but not in sCJD samples (n=8) when compared to non-CJD controls (n=8) (FIG. 11A).

Another MAb, 6H3, reacts with recombinant human PrP dimers and mouse PrP^{Sc} aggregates. Therefore, we investigated whether MAb 6H3 could detect human PrP^{Sc} aggregates. The results were similar to those obtained with MAb 11G5, except that the immunoreactivity detected with MAb 6H3 was much stronger (FIG. 1B).

The reasons that MAb 6H3 did not detect significant immunoreactivity in sCJD samples might be because sCJD samples had much less PrP^{Sc} aggregates, at levels below the detection limit of AS-ELISA. We, therefore, repeated the identical experiment with AS-FACT (FIG. 11C). As expected, the signals detected with either MAb 11G5 or MAb 6H3 were greatly magnified in vCJD brain homogenates. However, neither MAb 11G5 nor MAb 6H3 reacted significantly with sCJD samples, when compared to non-CJD controls.

To determine the sensitivity of the AS-FACT in detecting vCJD PrP^{Sc} aggregates, we serially diluted two of the vCJD samples and carried out AS-FACT using MAb 6H3. The assay could detect significant signals; even when the total brain protein concentrations were diluted to as low as 0.08 μg/ml. Therefore, the principle of AS-FACT is applicable to human vCJD, with high degrees of sensitivity and specificity.

Discussion

We developed a sensitive assay, AS-FACT for PrP^{Sc} detection by combining an aggregation-specific ELISA with a T7 polymerase amplification technique. Using MAb 11G5, the AS-FACT is able to detect PrP^{Sc} aggregates in the brain of majority of the animals as early as one week after an intraperitoneal inoculation. Thirty days after inoculation, all infected animals are positive. The principle of AS-FACT is also applicable to two other prion diseases, CWD in deer and elk, and vCJD in human.

While MAb 11G5 did react with CWD PrP^{Sc} in AS-ELISA, the signals were not as robust as mouse PrP^{Sc}; MAb 11G5 may not be the most sensitive MAb for detecting CWD PrP^{Sc} aggregates. Worthy of note is that while MAb, 11G5 detects mouse and CWD PrP^{Sc} aggregates, it does not react with vCJD aggregates. The detection of vCJD aggregates requires the use of a different MAb, 6H3. On the other hand, MAb 6H3 reacts with mouse PrP^{Sc} but not CWD PrP^{Sc} aggregates. These differences in immunoreactivities most likely reflect conformational differences between PrP^{Sc} aggregates in mouse, deer and human prion diseases.

One week after an intraperitoneal inoculation, PrP^{Sc} aggregates are detectable in the brains of most of the animals. Irrespective of the mechanisms of PrP^{Sc} transport, this finding suggests that some PrP^{Sc} aggregates are able to migrate to the CNS from peripheral tissue within one week. The immunoreactivity detected in these animals most likely is derived from the inoculants rather than de novo synthesized, host-derived PrP^{Sc}. This finding is consistent with an earlier study in hamsters using bioassays, which is currently the most sensitive assay for detecting infectious PrP^{Sc}. In this study, it was found that peripheral PrP^{Sc} could reach the CNS rather rapidly, within 10 days after an intraperitoneally inoculation. In another study, using the “Protein Misfolding Cyclic Amplification” (PMCA) technique, it was reported that PrP^{Sc} could be detected as early as two weeks after an intracerebral inoculation.

A conformational dependent immunoassay (CDI) has also been developed for PrP^{Sc} detection and strain identification. This assay is based on the principle that an epitope that is normally buried in PrP^{Sc} is exposed after treatment of the sample with denaturing agents. The sensitivity of CDI is about 10³ID₅₀/ml of PrPS. Direct comparison of these assays will be very informative.

In AS-FACT, MAb 6H3 is able to detect significant immunoreactivity in vCJD samples, even when the total brain protein concentrations were diluted to as low as 0.08 μg/ml. In terminally ill hamsters, there were about 100-1000 LD₅₀ units of infectivity per ng of brain proteins. If vCJD patients and hamsters have comparable levels of infectivity, AS-FACT potentially can detect between 160 and 1600 LD₅₀ units of infectivity. These numbers are most likely under estimated, because it is doubtful that the units of infectivity in vCJD patients could ever reach as high as that of infected hamsters.

On the other hand, neither 11G5 nor 6H3 react with PrP^{Sc} aggregates in sCJD. One possibility is that the PrP^{Sc} aggregates in sCJD have a different conformation. If this is the case, we may be able to identify a MAb that reacts with sCJD aggregates by screening more MAbs. All the human brain tissues used in this study were from the frontal cortex areas. Alternatively, the PrP^{Sc} aggregates being detected might be present in different brain regions in vCJD and sCJD. Both vCJD and the sCJD cases studied here belong to Type 2 PrP^{Sc}. Experiments are now in progress to determine whether the aggregation specific assays are applicable to other human CJD.

The precise natures of the PrP^{Sc} aggregates being detected with AS-ELISA are not known. Previous studies revealed that the immunoreactivity detected by AS-ELISA was PK resistant and had a molecular mass of approximately 2,000 KD. By ultracentrifugation in a sucrose gradient, most of the immunoreactivity detected by AS-ELISA is present in fractions 3, 4 and 5. These fractions are mostly devoid of PrP^{C} as it partitions to the top two fractions. On the other hand, the largest PrP^{Sc} aggregates tend to petition to the bottom fractions. Studies in hamster suggested that these smaller aggregates are infectious. The smaller PrP^{Sc} may represent the precursors of the larger aggregates. Alternatively, they may be the metabolic products of larger aggregates.

Currently, all the in vitro diagnostic tests for prion diseases require either the demonstration of PK-resistant PrP species in brain homogenate, in vitro amplification, or the uncovering of hidden epitopes after GdHC1 treatment. The aggregation specific assays described here provide certain advantages; 1) the assay may be more sensitive because of the use of a capture antibody and an amplification step; 2) with the use of more than one antibody, a built-in control can be used to monitor the specificity and sensitivity of the assay; 3) because the PrP^{Sc} aggregates being detected are smaller, they may be more likely to be present in the circulation or body fluids of infected animals or humans. Finally, the principle of the aggregate specific ELISA we have developed can be applicable to other diseases caused by abnormal protein aggregation, such as Alzheimer's disease or Parkinson's disease.

From the above description of the invention, those skilled in the art will appreciate improvements, changes, and modifications. Such improvements, changes and modifications are intended to be covered by the appended claims.

It will be appreciated that all patents listed above are herein incorporated by reference in their entirety, whether or not it is expressly stated.

Having described the invention, the following is claimed:

1. A method of detecting disease-related aggregates of proteins in a sample comprising:
preparing a first monoclonal antibody or an epitope-binding fragment thereof, which is immunoreactive with the protein aggregate;
bringing the first monoclonal antibody or epitope-binding fragment thereof into contact with the sample;
removing protein aggregate not bound by the first monoclonal antibody or epitope-binding fragment thereof;
bringing a second labeled monoclonal antibody or epitope-binding fragment thereof into contact with the protein aggregate bound by the first monoclonal antibody or epitope-binding fragment thereof, the second labeled monoclonal antibody or epitope-binding fragment thereof comprising a monoclonal antibody or epitope-binding fragment thereof identical to the first monoclonal antibody or epitope-binding fragment thereof; and
detecting the amount of second labeled monoclonal antibody or epitope-binding fragment thereof bound to the protein aggregate.

2. The method of claim 1, the first monoclonal antibody or epitope-binding fragment thereof being immobilized on a solid support.

3. The method of claim 2, further comprising incubating the first monoclonal antibody or epitope-binding fragment thereof and the sample before removing the protein aggregate not bound by the first monoclonal antibody or epitope-binding fragment thereof.

4. The method of claim 1, the second monoclonal antibody or epitope-binding fragment thereof being coupled to an RNA promoter-driven cDNA sequence.

5. The method of claim 1, the protein aggregate being at least one of beta-amyloid precursor protein (APP), beta-amyloid (βA), α-synuclein protein, tau protein, superoxide dismutase protein, Huntington protein, and prion protein (PrP).

6. The method of claim 5, the protein aggregate comprising of conformationally-altered prion protein.

7. The method of claim 1, the amount of second monoclonal antibody or epitope-binding fragment thereof bound to the aggregate being proportional to the amount of protein aggregate present in the biological sample.

8. A method of detecting disease related protein aggregate in a sample comprising:
preparing a first monoclonal antibody or an epitope-binding fragment thereof, which is immunoreactive with the protein aggregate;
bringing the first monoclonal antibody or epitope-binding fragment thereof into contact with the sample;
incubating the first monoclonal antibody or epitope-binding fragment thereof and the sample;
removing protein aggregate not bound by the first monoclonal antibody or epitope-binding fragment thereof;
bringing a second labeled monoclonal antibody or epitope-binding fragment thereof into contact with the protein aggregate bound by the first monoclonal antibody or epitope-binding fragment thereof, the second labeled monoclonal antibody or epitope-binding fragment thereof comprising a monoclonal antibody or epitope-binding fragment thereof identical to the first monoclonal antibody or epitope-binding fragment thereof; and
detecting the amount of second labeled monoclonal antibody or epitope-binding fragment thereof bound to the protein aggregate.

9. The method of claim 8, the first monoclonal antibody or epitope-binding fragment thereof being immobilized on a solid support.

10. The method of claim 8, the second monoclonal antibody or epitope-binding fragment thereof being coupled to an RNA promoter-driven cDNA sequence.

11. The method of claim 8, the protein aggregate being at least one of beta-amyloid precursor protein (APP), beta-amyloid (PA), α-synuclein protein, tau protein, superoxide dismutase protein, Huntington protein, and prion protein (PrP).

12. The method of claim 11, the protein aggregate comprising of conformationally-altered prion protein.

13. The method of claim 8, the amount of second monoclonal antibody or epitope-binding fragment thereof bound to the protein aggregate being proportional to the amount of protein aggregate present in the sample.

14. A method of detecting disease-related aggregate of prion protein in a sample comprising:
preparing a first monoclonal antibody or an epitope-binding fragment thereof, which is immunoreactive with prion protein;
bringing the first monoclonal antibody or epitope-binding fragment thereof into contact with the sample;
removing prion protein not bond to the first monoclonal antibody or epitope-binding fragment thereof;
bringing a second labeled monoclonal antibody or epitope-binding fragment thereof into contact with the prion protein bound to the first monoclonal antibody or epitope-binding fragment thereof, the second labeled monoclonal antibody or epitope-binding fragment thereof comprising a monoclonal antibody or epitope-binding fragment thereof identical to the first monoclonal antibody or epitope-binding fragment thereof; and
detecting the amount of second labeled monoclonal antibody or epitope-binding fragment thereof bound to the prion protein.

15. The method of claim 14, the first monoclonal antibody or epitope-binding fragment thereof being immobilized on a solid support.

16. The method of claim 14, further comprising incubating the first monoclonal antibody or epitope-binding fragment thereof and the sample before removing the prion protein not bond to the first monoclonal antibody or epitope-binding fragment thereof.

17. The method of claim 1, the second monoclonal antibody or epitope-binding fragment thereof being coupled to an RNA promoter-driven cDNA sequence.

18. The method of claim 14, the amount of second monoclonal antibody or epitope-binding fragment thereof bound to the prion protein being proportional to the amount of protein aggregate present in the biological sample.
